(19) United States
(12) Patent Application Publication
(10) Pub. No.: US 2017/0232151 A1
(43) Pub. Date: Aug. 17, 2017

(54) BIORESORBABLE SYNTHETIC BONE GRAFT

(71) Applicants: National Taiwan University, Taipei (TW); Chang Gung Memorial Hospital, Linkou, Taoyuan (TW)

(72) Inventors: WEI-HSING TUAN, Taipei (TW); PO-LIANG LAI, Taoyuan (TW); MAN-PING CHANG, Taipei (TW); CHIA-JUNG WU, Taoyuan (TW); HSIU-CHING HSU, Taipei (TW)

(21) Appl. No.: 15/428,159

(22) Filed: Feb. 9, 2017

Related U.S. Application Data
(60) Provisional application No. 62/295,013, filed on Feb. 13, 2016.

(51) Int. Cl. A61L 27/58; A61L 27/20; A61L 27/02

(52) U.S. Cl. CPC A61L 27/58; A61L 27/025; A61L 27/20; A61L 2430/02; A61L 2400/06

(57) ABSTRACT

A solid solution for use in bone regeneration. The solid solution includes two divalent cations, wherein a first divalent cation is calcium ion (Ca²⁺) and a second divalent cation is selected from the group consisting of magnesium ion (Mg²⁺), zinc ion (Zn²⁺), barium ion (Ba²⁺) and strontium ion (Sr²⁺). The solid solution also includes at least one anion, and the at least one anion comprises one or more of sulfate (SO₄²⁻), phosphate (PO₄²⁻), carbonate (CO₃²⁻), and silicate (SiO₃²⁻).

### RELATED APPLICATIONS

This application claims priority to U.S. Provisional Application No. 62/295,013, filed on Feb. 13, 2016, which is hereby incorporated by reference in its entirety.

### FIELD

The present disclosure relates to a bioresorbable synthetic bone graft. The bioresorbable synthetic bone graft can be implanted in traumatized or osteoporotic fracture sites in the bone. More particularly, the present disclosure provides a solid solution for use in bone regeneration, the solid solution comprises two divalent cations and at least one anion.

### BACKGROUND

Osteoporosis is a bone disease characterized by low bone mass and the deterioration of density in bone. For patients suffering from osteoporosis, lowered rate of bone formation (osteogenesis) and increased rate of bone loss lead to decline in bone mass and density, thus increase the chance of developing consequent bone fractures. Osteoporosis is more frequently occurred in elderly people and postmenopausal women.

Other bone disease related to low bone mass include inadequate response to bone fractures (e.g., delayed union, nounion or malunion), arthrodesis, cystic defects and skeletal defects after tumor removal.

Some medications have been approved by the FDA to treat osteoporosis. Available medications achieve its antiosteoporotic effects by modulating bone formation or bone loss of osteoporotic sites of the bone. The underlying mechanisms of these medications are: i) anti-resorptive agents: the anti-resorptive agents modulate the rate of bone loss and reduce bone remodeling, available anti-resorptive agents may include: bisphosphates (e.g., Alendronate, Ibandronate, Risedronate and Zoledronic acid), hormone replacements (e.g., estrogen), selective estrogen receptor modulators (SERMs), calcitonin and RANKL inhibitors (e.g., Denosumab); ii) osteogenic agents: the osteogenic agents facilitate the rate of bone formation, available osteogenic agents include recombinant parathyroid hormone, and iii) mixed type medications: mixed type medications modulate the rate of bone formation and inhibit bone loss simultaneously, the only available mixed type medication is strontium ranelate (C₁₂H₆N₂O₈SSr₂).

Approved medications for treating osteoporosis are mostly delivered orally or intravenously. Some medications are delivered by nasal spray, transdermal patch, subcutaneous injection or intramuscular injection. Antiosteoporotic medications requires a stringent medication schedule for a long time, because osteoporotic fracture sites of the bone may take several months to recover. The medications for treating osteoporosis are sometimes inefficient due to insufficient drug absorption or poor patient compliance.

An alternative therapeutic approach to treat bone diseases related to bone deficiency is to implant bone grafts to osteoporotic fracture sites. Bone graft is a surgical implant intends to fill, augment, or reconstruct bone defects. Bone void filler is a bone graft intends to fill the voids or gaps of bones caused by trauma, surgery or other defects that would affect the stability of the bone structure. The bone void filler is implanted into the location of defects to assist the repair and regeneration.

An ideal bone void filler should possess both osteoconduction and osteoinduction properties. Osteoconduction refers to the attachment, migration, and growth of osteoblasts, chondroblasts, or other osteogenic precursor cells on 3-dimensional scaffolds in the implantation site. Osteoinduction refers to the environment for which osteogenic precursor cells can be recruited and differentiated.

Bioceramic materials are often being used as bone void fillers, such as hydroxyapatite (Ca₁₀(PO₄)₆(OH)₂), calcium sulfate crystals (e.g., CaSO₄.2H₂O, CaSO₄0.5H₂O), calcium carbonate (CaCO₃), calcium phosphates (e.g., CaPO₄, Ca(H₂PO₄)₂, CaHPO₄, Ca₃(PO₄)₂, Ca₄(PO₄)₂O, Ca₈H₂(PO₄, Ca₅(PO₄)₃(OH) and calcium oxide (CaO). After implantation into the bone, the bioceramic bone void filler is degraded and resorbed by human body, thus releases the calcium ion (Ca²⁺) to promote osteogenesis. FDA approved bioceramic bone void filler products include Osteoset®, Bone Plast™, Bone Source®, a-BEM®, Bioborn® and Vitoss®.

Silicon compounds or sodium compounds can be mixed with bioceramic to facilitate osteogenesis of osteoporotic fracture sites. The silicon compounds or sodium compounds may include but not limited to silicon dioxide (SiO₂), sodium hydrogen phosphate (Na₂HPO₄) and sodium oxide (Na₂O). Phosphorous pentoxide (P₂O₅) may also be mixed with the above silicon compounds and bioceramic. FDA approved bioceramic bone void filler products containing silicon compound or sodium compound include Calcibon®, Bioglass® and Skeletal Repair System™.

Organic materials can be mixed with bioceramic to optimize the osteoinduction of the bone void filler. Components of extracellular matrix of bone tissue would be organic materials suitable for mixing with biocermic to create an osteoinductive environment for osteogenic precursor cells. The organic materials may include but not limited to collagen, agarose gel, carrageenan or gelatin. Collagraft® is a FDA approved bone void filler product contains bioceramic and collagen.

However, the rate of releasing calcium ion (Ca²⁺) in most of the bioceramic bone grafts is too high. The osteoporotic fracture sites or traumatized sites in the bone often take several months to heal and regenerate, but most of the current bioceramic bone grafts degraded faster than the time required for bone regeneration. This may lead to decreased strength in bone tissues due to incomplete bone regeneration.

Sintered calcium compounds are used as a bone graft to adjust the rate of releasing calcium ion (Ca²⁺). Sintering refers to the process of elevating the temperature of a solid material without reaching its melting point. Sintered material generally has a lower surface area and a higher density comparing to non-sintered material of the same compound. A reduced surface area results in slower degradation or dissolution rate, which affects the rate of releasing calcium ion (Ca²⁺).

U.S. Pat. No. 5,462,722 disclosed a composite of calcium sulfate (CaSO₄) material and calcium phosphate (CaPO₄), wherein calcium sulfate (CaSO₄) material is sintered first and mixed with calcium phosphate (CaPO₄) later. U.S. Patent Publication No. 20030055511 disclosed a calcium sulfate (CaSO₄) material or calcium phosphate (CaPO₄) material for repairing a bone deficiency, wherein the calcium sulfate (CaSO₄) material or calcium phosphate (CaPO₄) material can be sintered. U.S. Pat. No. 7,417,077 disclosed a bone mineral substitute material, which is a sintered mixture of calcium sulfate hemihydrate (CaPO₄ 0.5H₂O) and hydroxyapatite (Ca₁₀(PO₄)₆(OH)₂). U.S. Patent Publication No. 20040002770 disclosed a porous bioceramic host, wherein the bioceramic host is a composite of polymers and sintered bioceramic materials, wherein the sintered bioceramic materials is an inorganic salt consisting of calcium ion (Ca²⁺) as cation, and sulfate, phosphate or carbonate ions as anions. U.S. Pat. No. 8,263,513 and U.S. Pat. No. 8,906,817 disclosed a sintered calcium sulfate (CaSO₄) material for use in the bone regeneration.

U.S. Patent Publication No. 20080317807 disclosed a strontium-fortified calcium nanoparticles or microparticles, wherein the nanoparticles or microparticle is electrophoretically deposit onto a bone implant, and the bone implant is sintered at a temperature less than 800° C.

It would be desirable to optimize the rate of releasing calcium ion (Ca²⁺) of bioceramic materials by sintering. It would be also desirable to release antiosteoporotic agents locally in the osteoporotic fracture sites. Therefore, it is an object of the present disclosure to provide a sintered bioceramic material having a slower rate of releasing calcium ion (Ca²⁺), and releasing at least one antiosteoporotic agent when implanted into the bone to facilitate bone regeneration.

It is also an object of the present disclosure to provide a bioresorbable bone graft comprising a homogeneous solid solution containing a solid solution with a host of calcium compounds and a solute of antiosteoporotic agents.

It is also an object of the present disclosure to provide a bioresorbable bone graft comprising a solid solution containing two divalent cations and at least one anion.

It is also an object of the present disclosure to provide a bioresorbable bone graft composed of a sintered antiosteoporotic agent.

### BRIEF DESCRIPTION OF THE DRAWINGS

The present description will be better understood from the following detailed description read in light of the accompanying drawings, where:

FIG. 1A illustrates the schematic of the grains in a mixture of two materials in accordance with aspects of present disclosure.

FIG. 1B illustrates the schematic of the grains in a solid solution in accordance with aspects of present disclosure.

FIG. 2A illustrates the schematic of a mixture of two materials in accordance with aspects of present disclosure.

FIG. 2B illustrates the schematics of a solid solution in accordance with aspects of present disclosure.

FIG. 3 illustrates the flowchart for preparing the bioresorbable bone graft pellets comprising a solid solution in accordance with aspects of present disclosure.

FIG. 4 illustrates the flowchart of the solubility test of the bioresorbable bone graft pellets comprising a solid solution in accordance with aspects of present disclosure.

FIG. 5A, FIG. 5B and FIG. 5C are the remains of the sintered powder in the buffered citric acid solution at the bottom of the plastic bottle after the extreme test in accordance with aspects of present disclosure.

FIG. 6 illustrates the weight loss of the sintered pellets comprising a solid solution after the extreme test and the simulation test in accordance with aspects of present disclosure.

FIG. 7 illustrates the pH value of the solutions in the extreme test and the simulation test for the sintered pellets comprising a solid solution in accordance with aspects of present disclosure.

FIG. 8 illustrates the pH value of the solutions during the degradation test for CS-1100 and Sr-CS-1200 in accordance with aspects of present disclosure.

FIG. 9 illustrates the accumulated weight loss of CS-1100 and Sr-CS-1200 during the degradation test in accordance with aspects of present disclosure.

FIG. 10 illustrates the concentration of calcium ion (Ca²⁺) released by CS-1100 and Sr-CS-1200 during the degradation test in accordance with aspects of present disclosure.

FIG. 11 illustrates the concentration of strontium ion (Sr²⁺) released by Sr-CS-1200 during the degradation test in accordance with aspects of present disclosure.

FIG. 12 illustrates the cell viability of the cytotoxicity test on extracts of CS-1100 in accordance with aspects of present disclosure.

FIG. 13 illustrates the cell viability of the cytotoxicity test on extracts of Sr-CS-1200 in accordance with aspects of present disclosure.

FIG. 14 illustrates the cell viability of the cytotoxicity test on extracts of Sr-1000 in accordance with aspects of present disclosure.

FIG. 15A, FIG. 15B, FIG. 15C, FIG. 15D, FIG. 15E and FIG. 15F are MC3T3-E1 cell morphologies under 400× magnification by confocal microscopy after direct contact with CS-1100, Sr-CS-1200 and Sr-1000 for 24 hours in accordance with aspects of present disclosure.

FIG. 16A, FIG. 16B, FIG. 16C, FIG. 16D, FIG. 16E and FIG. 16F are MC3T3-E1 cell morphologies under 400× magnification by confocal microscopy after direct contact with CS-1100, Sr-CS-1200 and Sr-1000 for 72 hours in accordance with aspects of present disclosure.

FIG. 17A, FIG. 17B and FIG. 17C are the surface images of CS-1100, Sr-CS-1200 and Sr-1000 under 1000× magnification by scanning electronic microscopy after direct contact with MC3T3-E1 cells for 24 hours in accordance with aspects of present disclosure.

FIG. 18A, FIG. 18B and FIG. 18C are the surface images of CS-1100, Sr-CS-1200 and Sr-1000 under 1000× magnification by scanning electronic microscopy after direct contact with MC3T3-E1 cells for 72 hours in accordance with aspects of present disclosure.

FIG. 19 illustrates the relative densities of calcium sulfate hemihydrate (CaSO₄ 0.5H₂O) pellets after sintered at different temperatures in accordance with aspects of present disclosure.

FIG. 20 illustrates the relative densities of strontium-calcium solid solution pellets formed by sintering at different temperatures in accordance with aspects of present disclosure.

FIG. 21 illustrates the relative densities of strontium sulfate (SrSO₄) pellets after sintered at different temperatures in accordance with aspects of present disclosure.

FIG. 22 is the X-ray diffraction patterns of calcium sulfate hemihydrate (CaSO₄ 0.5H₂O) pellets after sintered at different temperatures in accordance with aspects of present disclosure.

FIG. 23 is the X-ray diffraction patterns of strontium-calcium solid solution pellets formed by sintering at different temperatures in accordance with aspects of present disclosure.

FIG. 24 is the X-ray diffraction patterns of strontium sulfate (SrSO₄) pellets after sintered at different temperatures in accordance with aspects of present disclosure.

FIG. 25A, FIG. 25B and FIG. 25C are the surface of CS-1100, Sr-CS-1200 and Sr-1000 under 1000× magnification by scanning electronic microscopy in accordance with aspects of present disclosure.

FIG. 26 is the appearance of rat calvarial bone when implanted with Sr-CS-1200 and Sr-1000 in accordance with aspects of present disclosure.

FIG. 27A, FIG. 27B, FIG. 27C, FIG. 27D, FIG. 27E and FIG. 27F are the X-ray images of rat calvarial bone implanted with Sr-CS-1200 and Sr-1000 after 2 weeks, 8 weeks, 12 weeks, 16 weeks, 20 weeks and 24 weeks, in accordance with aspects of present disclosure.

FIG. 28 is the appearance of bioresorbable bone graft paste containing Sr-CS-1100, in accordance with aspects of present disclosure.

FIG. 29 is the appearance of bioresorbable bone graft paste containing Sr-CS-1100 in the water, in accordance with aspects of present disclosure.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Bioresorbable bone grafts intend to release cations in the human body once implanted in the osteoporotic fracture sites of traumatized sites, and the cations may include but not limited to calcium ion (Ca²⁺), strontium ion (Sr²⁺), magnesium ion (Mg²⁺), zinc ion (Zn²⁺), barium ion (Ba²⁺) and any other cations that assists bone regeneration. The bioresorbable bone grafts or bone void fillers may contain bioceramic materials, and bioceramic materials release calcium ion (Ca²⁺) by degradation in the human body. In the present disclosure, by using a solid solution in a bioresorbable bone graft, the rate of degradation and dissolution are optimized, therefore the rate of releasing calcium ion (Ca²⁺) is slower. It is also desirable to release antiosteoporotic agents to the osteoporotic fracture sites by incorporating at least one strontium compound in the solid solution of the bioresorbable bone graft. Thus, the present disclosure is directed to an improved treatment of osteoporosis by the direct release of strontium ion (Sr²⁺) and calcium ion (Ca²⁺) to the bone.

The present disclosure is directed to a solid solution for use in bone regeneration. The solid solution includes a host comprising at least one calcium compound, and a solute comprising at least one strontium compound, and the solid solution is bioresorbable. The calcium compound can be any one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), calcium oxide (CaO) and calcium silicate (CaSiO₃). The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The solid solution is sintered at a given temperature, the given temperature is ranged from about 800° C. to 1300° C. The molar percentage of strontium compound in the solid solution is about 1% to 50%.

According to one or more exemplary embodiments, the solid solution comprises calcium sulfate anhydrite (CaSO₄) and strontium sulfate (SrSO₄). The molar percentage of strontium sulfate (SrSO₄) in the solid solution is about 7%. The solid solution is sintered at 1200° C.

The present disclosure further is directed to a solid solution for use in bone regeneration. The solid solution includes two divalent cations, wherein a first divalent cation is calcium ion (Ca²⁺) and a second divalent cation is selected from the group consisting of magnesium ion (Mg²⁺), zinc ion (Zn²⁺), barium ion (Ba²⁺) and strontium ion (Sr²⁺). The solid solution further includes at least one anion, and the at least one anion comprises one or more of sulfate (SO₄²⁻), phosphate (PO₄²⁻), carbonate (CO₃²⁻) and silicate (SiO₃²⁻). The molar ratio of the two divalent cations to the anion is 1 to 1.5. The relative density of the solid solution is higher than 65%.

The present disclosure further is directed to a bioresorbable bone graft pellets for use in bone regeneration. The bioresorbable bone graft pellets include a solid solution. The solid solution is having at least one strontium compound and at least one calcium compound. The calcium compound can be any one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (C₁₂H₆N₂O₈SSr₂), hydroxyapatite (Ca₅(PO₄)₃(OH), calcium carbonate (CaCO₃), calcium oxide (CaO) and calcium silicate (CaSiO₃). The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The solid solution is sintered at a given temperature, the given temperature is ranged from about 800° C. to 1300° C. The molar percentage of strontium compound in the solid solution is about 1% to 50%.

According to one or more exemplary embodiments, the solid solution of the bioresorbable bone graft pellet comprises calcium sulfate anhydrite (CaSO₄) and strontium sulfate (SrSO₄). The molar percentage of strontium sulfate (SrSO₄) in the solid solution is about 7%. The solid solution is sintered at 1200° C.

The present disclosure further is directed to a bioresorbable bone graft product for use in bone regeneration. The bioresorbable bone graft product includes a powder mixture, a binder, and a liquid. The powder mixture comprises a solid solution powder, and the solid solution powder is having at least one strontium compound and at least one calcium compound. The calcium compound can be any one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), calcium oxide (CaO) and calcium silicate (CaSiO₃). The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The solid solution powder is sintered at a given temperature, the given temperature is ranged from about 800° C. to 1300° C. The molar percentage of strontium sulfate in the solid solution powder is about 1% to 50%. The powder mixture further comprises a hardening agent, and the hardening agent can be any one or more of calcium sulfate anyhydrate (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), magnesium carbonate (MgCO₃), strontium carbonate (SrCO₃), sodium hydrogen phosphate (Na₂HPO₄), and the bioglass. The binder can be a synthetic organic polymer or a natural organic polymer. The natural organic polymer comprises one or more of agarose gel, alginate, carrageenan, chitosan, collagen, fibrinogen, gelatin, hyaluronic acid, and starch. The synthetic organic polymer comprises one or more of polyactic acid (PLA), poly(lactic-co-glycolic acid) (PLGA), poly-L-lactide (PLLA), poly-DL-lactic acid (PDLLA), polycaprolactone (PCL), polyethylene glycol, poly(α-hydroxy ester), poly(N-isopropryl acrylamide), pluronic block copolymers, and carboxymethyl cellulose. The liquid can also be any one of animal serum, human serum, human blood, human bone marrow aspirate, Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid.

The present disclosure further is directed to a method for preparing and implanting bioresorbable bone graft paste. The bioresorbable bone graft paste is prepared by mixing materials in a bioresorbable bone graft product. The materials includes a powder mixture, a binder, and a liquid. The method includes: a) mix the powder mixture, the binder and the liquid, thereby generating a bioresorbable bone graft paste; b) deliver the bioresorbable bone graft paste in the implantation location.

The present disclosure further is directed to a bioresorbable bone graft product for use in bone regeneration. The bioresorbable bone graft product includes a solid solution powder, a binder, and a liquid. The solid solution powder is having at least one strontium compound and at least one calcium compound. The calcium compound can be any one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), calcium oxide (CaO) and calcium silicate (CaSiO₃). The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The solid solution is sintered at a given temperature, the given temperature is ranged from about 800° C. to 1300° C. The molar percentage of strontium sulfate in the solid solution is about 1% to 50%. The binder can be an organic polymer. The organic polymer is a synthetic organic polymer or a natural organic polymer. The natural organic polymer comprises one or more of agarose gel, alginate, carrageenan, chitosan, collagen, fibrinogen, gelatin, hyaluronic acid, and starch. The synthetic organic polymer comprises one or more of polyactic acid (PLA), poly(lactic-co-glycolic acid) (PLGA), poly-L-lactide (PLLA), poly-DL-lactic acid (PDLLA), and polycaprolactone (PCL), polyethylene glycol, poly(α-hydroxy ester), poly(N-isopropryl acrylamide), pluronic block copolymers, and carboxymethyl cellulose. The liquid can be any one of animal serum, human serum, human blood, human bone marrow aspirate, Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid.

The present disclosure further is directed to a bioceramic material for use in bone regeneration. The bioceramic material is a solid solution. The solid solution is having at least one calcium compound and at least one strontium compound. The calcium compound can be any one or more of calcium sulfate anhydrite (CaSO₄), calcium sulfate hemihydrate (CaSO₄ 0.5H₂O), calcium sulfate dihydrate (CaSO₄. 2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), calcium oxide (CaO) and calcium silicate (CaSiO₃). The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The solid solution is sintered at a given temperature, the given temperature is ranged from about 800° C. to 1300° C. The molar percentage of strontium compound in the solid solution is about 1% to 50%.

According to one or more exemplary embodiments, the solid solution of the bioceramic material comprises calcium sulfate anhydrite (CaSO₄) and strontium sulfate (SrSO₄). The molar percentage of strontium sulfate (SrSO₄) in the solid solution is about 7%. The solid solution is sintered at 1200° C.

The present disclosure further is directed to a method for implanting a bioresorbable bone graft. The method includes: a) identify the location of bone defect; b) prepare the location of bone defect; c) deliver the bioresorbable bone graft. The bioresorbable bone graft includes a bioceramic material. The bioceramic material is a solid solution. The solid solution is a combination of at least one calcium compound and at least one strontium compound. The calcium compound can be any one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), calcium oxide (CaO) and calcium silicate (CaSiO₃). The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The solid solution is sintered at a given temperature, the given temperature is ranged from about 800° C. to 1300° C. The molar percentage of strontium compound in the solid solution is about 1% to 50%. The bioresorbable bone graft is in the form of pellet or paste.

According to one or more exemplary embodiments, the solid solution of the bioceramic material for use in the method for implanting a bioresorbable bone graft comprises calcium sulfate anhydrite (CaSO₄) and strontium sulfate (SrSO₄). The molar percentage of strontium sulfate (SrSO₄) in the solid solution is about 7%. The solid solution is sintered at 1200° C.

According to one or more exemplary embodiments, the bioresorbable bone graft used in the method can be in the form of pellet or paste.

The present disclosure further is directed to a bioresorbable bone graft for use in bone regeneration. The bioresorbable bone graft comprises a strontium compound. The strontium compound can be any one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The bioresorbable bone graft is in the form of pellets or paste. The strontium compound is sintered at about 800° C. to 1300° C.

According to one or more exemplary embodiments, the sintered material of the bioresorbable bone graft is strontium sulfate (SrSO₄). The sintered material is sintered at 1000° C.

The present disclosure further is directed to a solid solution for use in bone regeneration. The solid solution comprises two divalent cations, wherein a first divalent cation is strontium (Sr²⁺) and a second divalent cation is selected from the group consisting of magnesium (Mg²⁺), zinc (Zn²⁺), and barium (Ba²⁺). The solid solution further includes at least one anion, and the at least one anion comprises one or more of sulfate (SO₄²⁻), phosphate (PO₄²⁻), carbonate (CO₃²⁻) and silicate (SiO₃²⁻). The molar ratio of the two divalent cations to the anion is 1 to 1.5. The relative density of the solid solution is about 65% to 100%.

The term “bioresorbable” as used herein refers to a property of the material, which the material can be degraded and broken down in the animal body, human body or ex-vivo cell cultures without being mechanically removed. The bioresorbable behavior of a material can be observed in in-vitro dissolution or degradation tests, and the in-vitro dissolution or degradation tests may use solutions to simulate the chemical properties inside human body. The solutions for in vitro dissolution or degradation tests may include, but not limited to TRIS-HCl buffer solution, citric acid solution, Hank's balanced salt solution, phosphate buffered saline, sodium hydrogen phosphate (Na₂HPO₄) solution, distilled water, or stimulated body fluid. The term “bone graft” as used herein refer to the surgical implant intends to fill, augment, or reconstruct bone defects. The term “bone void filler” as used herein refer to a bioresorbable bone graft intends to fill the voids or gaps of bones caused by trauma, surgery or other defects that would affect the stability of the bone structure. The term “sintering” as used herein refers to the process of heat treating a solid material without reaching its melting point. The term “sintered” as used herein refers to a condition of material which the sintering has been utilized on the material.

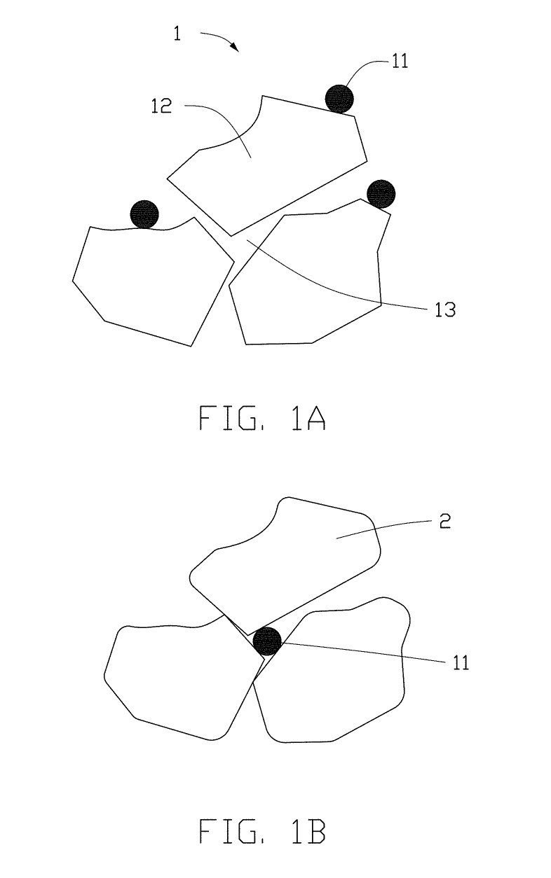

FIG. 1A is an illustration of the schematics of the grains in a mixture. Mixture 1 is a mixture of compound 11 and compound 12. As illustrated in FIG. 1A, the grain size of compound 12 is significantly larger than the grain size of compound 11. Pore 13 is formed at the grain boundaries of compound 12.

FIG. 1B is an illustration of the schematics of the grains in a solid solution. The solid solution is a solid solution that contains two or more chemical species. The solid solution 2 is consisting of compound 11 and compound 12, and compound 11 can be seen as a solute and compound 12 can be seen as a host. Compound 11 is incorporated into pore 13 formed at the grain boundaries of compound 12. The driving force to move compound 11 into pore 13 can be heat treatment. The elevated temperature reduces the grain boundaries of a solid material, as seen in FIG. 1A and FIG. 1B. The reduction of grain boundaries leads to a reduced surface area. Mixture 1 and solid solution 2 are composed of compound 11 and compound 12, but solid solution 2 is more compact than mixture 1 due to reduced surface area. Consequently, solid solution 2 may have a lower solubility in water than mixture 1.

Solid solution 2 can be a bioceramic material, and the bioceramic material can be used as a bone graft. The lowered solubility may lead to a slower rate of releasing calcium ion (Ca²⁺), thus bone regeneration may be better coordinated by the bone graft containing solid solution 2.

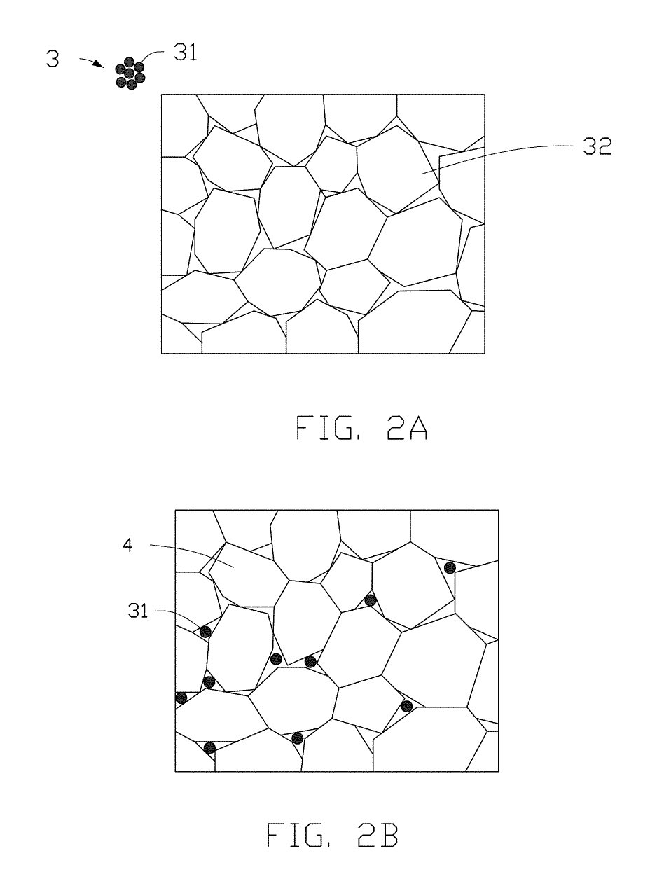

FIG. 2A is an illustration of the schematics of a mixture of two materials. Mixture 3 is a blend of compound 31 and compound 32, and compound 31 does not incorporated into the grain boundaries of compound 32.

FIG. 2B illustrates of the schematics of a solid solution. Solid solution 4 is consisting of compound 31 and compound 32, and compound 31 can be seen as a solute and compound 32 can be seen as a host. Compound 31 is incorporated into compound 32, forming a solid solution 4. Despite having the same composition, the surface area of solid solution 4 is smaller than mixture 3 because of the sintering. Some pores remain within the solid solution 4, and some compound 31 do not incorporate into compound 32. The density of solid solution 4 is larger than mixture 3 because the sintering has made solid solution 4 more compact than mixture 3. The solubility of solid solution 4 in a particular liquid may be also lower than mixture 3. The particular liquid may be body fluids containing cells, body fluids without cells, inorganic solutions or water. The particular liquid may include but not limited to human blood, human serum, animal serum, human bone marrow aspirate, TRIS-HCl buffer solution, citric acid solution, Hank's balanced salt solution, phosphate buffered saline, sodium hydrogen phosphate (Na₂HPO₄) solution, water, and stimulated body fluid. The solubility of solid solution 4 and mixture 3 in a particular liquid can be measured in accordance with ISO 10993-14 protocols.

Compound 31 is homogeneously dissolved in compound 32 to form solid solution 4 in FIG. 2B. The solid solution 4 is a homogeneous crystalline phase. The homogeneous dissolution of compound 31 is the result of sintering or heat treating, therefore solid solution 4 may be formed by sintering mixture 3. The homogeneous dissolution of compound 31 is reached by sintering at a given temperature, the given temperature is ranged from 800° C. to 1300° C. Most of compound 31 is incorporated into the grains of compound 32, therefore only few free compound 31 remains after the sintering. Due to the homogeneous dissolution of compound 31, the cation of compound 31 can be jointly released with the cation of compound 32 when solid solution 4 is dissolved in a particular liquid or when solid solution 4 is implanted in a human body or animal body. Compound 31 is a solid material having a much lower solubility in the particular liquid than compound 32. Compound 31 can be a strontium compound, including but not limited to: strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). Compound 32 can be a calcium compound. The calcium compound is a solid at room temperature and normal atmospheric pressure, including but not limited to: calcium sulfate anhydrite (CaSO₄), calcium sulfate hemihydrate (CaSO₄0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), calcium oxide (CaO) and calcium silicate (CaSiO₃).

If compound 31 is a strontium compound and compound 32 is a calcium compound, then the solid solution 4 comprises two divalent cations and at least one anion, wherein a first divalent cation is calcium ion (Ca²⁺) from compound 32, and the second divalent cation is strontium ion (Sr²⁺) from compound 31. The molar ratio of the first divalent cation to the second divalent cation is about 1 to 33, wherein the ratio of moles of the first divalent cation to the second divalent cation can be 1:1 to 97:3. The anion may be sulfate (SO₄²⁻), phosphate (PO₄²⁻), carbonate (CO₃²⁻) or silicate (SiO₃²⁻) from compound 31 and compound 32. The molar ratio of the two divalent cations to the anion is about 1 to 1.5, such as 1, 1.1, 1.2, 1.3, 1.4, or 1.5.

Alternatively, compound 31 can be another divalent cation releasing compound other than strontium compound. The divalent cations other than strontium ion (Sr²⁺) are also beneficiary to bone regeneration. Compound 31 can be a magnesium compound, a zinc compound or a barium compound, including but not limited to: magnesium sulfate (MgSO₄), magnesium phosphate (MgPO₄), zinc sulfate (ZnSO₄), zinc phosphate (Zn₃(PO₄)₂), barium sulfate (BaSO₄) and barium phosphate (Ba₃(PO₄)₂).

Alternatively, compound 32 can be a strontium compound, including but not limited to: strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂).

If compound 32 is a strontium compound and compound 31 is a magnesium compound, zinc compound or barium compound, then solid solution 4 contains two divalent cations, wherein a first divalent cation is strontium ion (Sr²⁺) from compound 32, and the second divalent cation is a magnesium ion (Mg²⁺), zinc ion (Zn²⁺) or barium ion (Ba2+). The molar ratio of the first divalent cation to the second divalent cation is about 1 to 33, wherein the ratio of moles of the first divalent cation to the second divalent cation can be 1:1 to 97:3. The anion may be sulfate (SO₄²⁻), phosphate (PO₄²⁻), carbonate (CO₃²⁻) or silicate (SiO₃²⁻) from compound 31 and compound 32. The molar ratio of the two divalent cations to the anion is about 1 to 1.5, such as 1, 1.1, 1.2, 1.3, 1.4, or 1.5.

FIG. 2B is a theoretical schematic of a solid solution formed by sintering or heat treating mixture 3. However, solid solution 4 may contain other crystal forms of compound 32. There is possible phase transformations of compound 32 during the sintering or heat treating of mixture 3, and compound 32 may be partly or entirely transformed into another crystal form after the sintering or heat treating. After the phase transformation, compound 32 may have a major crystal form, and one or more minor crystal forms. The major crystal form may constitute for more than 95% of the molar percentage of compound 32, and the minor crystal forms may constitute for less than 5% of the molar percentage of compound 32.

According to one or more exemplary embodiments of the present disclosure, compound 32 before the sintering can be calcium sulfate hemihydrate (CaSO₄0.5H₂O), calcium sulfate dihydrate (CaSO₄.2H₂O) or octacalcium phosphate (Ca₈H₂(PO₄)₆.5H₂O). After the sintering, the water molecules in calcium sulfate hemihydrate (CaSO₄ 0.5H₂O), calcium sulfate dihydrate (CaSO₄.2H₂O) or octacalcium phosphate (Ca₈H₂(PO₄)₆.5H₂O) are removed, and the major crystal form can be calcium sulfate anhydrite (CaSO₄). The minor crystal forms can be calcium sulfate hemihydrate (CaSO₄ 0.5H₂O), calcium sulfate dihydrate (CaSO₄.2H₂O), octacalcium phosphate (Ca₈H₂(PO₄)₆.5H₂O), or combinations thereof.

A bioresorbable bone graft may comprise compound 31 or compound 32. When compound 31 or compound 32 is individually implanted as separate bone grafts, implanted compound 31 has a much lower degradation rate than implanted compound 32. Therefore, the cations of compound 31 has a slower release rate in human body than the cations of compound 32.

A bioresorbable bone graft may comprise solid solution 4, and solid solution 4 comprises compound 31 and compound 32. When solid solution 4 is implanted in the human body, the cation of compound 31 is jointly released with the cation of compound 32. The homogeneous dissolution of compound 31 in solid solution 4 has led to an optimized degradation rate due to a higher relative density. The relative density is percentage of the measured density of the material to the theoretical density of the same material. Higher relative density suggests the material has fewer grain boundaries and the material is more condensed. If the solid solution 4 is composed of 7% compound 31 and 93% compound 32 by molar percentage, wherein compound 31 is a strontium compound and the compound 32 is a calcium compound, the relative density is about 80% to 95%, and the degradation rate of the bioresorbable bone graft comprising solid solution 4 is about 0.1% to 3% of the weight percentage per day.

A bioresorbable bone graft comprising solid solution 4 may be in the form of pellets. Compound 31 and compound 32 are mixed, and the mixture containing compound 31 and compound 32 is pelletized. The pellets of the mixture containing compound 31 and compound 32 are then sintered to form a solid solution 4. A bioresorbable bone graft pellets comprising solid solution 4 is implanted as a solid material to the osteoporotic fracture site or traumatized site of the bone. The pellets may be 3 mm to 7 mm in diameter. If the voids, gaps or spaces in the bone are smaller than the size of the pellets, the pellets would be difficult to enter the voids, gaps or spaces of the bone, thereby affecting the bone regeneration progress.

The bioresorbable bone graft comprising solid solution 4 may be in the form of paste. The bioresorbable bone graft paste comprising solid solution 4 is implanted as a plastic material to fill in voids, gaps or spaces smaller than 3 mm in diameter in the bone. The solid solution 4 may be in the form of powder when provided to the surgeon before the surgery. The powder of solid solution 4 is then mixed with a binder and a liquid to form the bioresorbable bone graft paste. The bioresorbable bone graft paste can be filled in a sterilized container, the sterilized container may be a syringe, a cup or any other containers suitable for containing a paste. The bioresorbable bone graft paste is then implanted in the human body by the surgeon. The paste form bioresorbable bone graft will remain plastic within at least 20 minutes after the implantation.

The liquid reacts with the calcium ion (Ca²⁺) in the bioresorbable bone graft to transform the bioresorbable bone graft into a rigid material when the bioresorbable bone graft paste is implanted. The liquid can be derived from biological origin or non-biological origin. The liquid derived from biological origin is a liquid derived from animal or human body. The liquid derived from biological origin can be any one of animal serum, human serum, human blood, and human bone marrow aspirate. The liquid derived from non-biological origin is a liquid not directly obtained from animal or human body, and the liquid derived from non-biological origin can be any one of Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid. The weight percentage of the liquid in the paste form bioresorbable bone graft may be about 20% to 70%.

The binder is mixed with the solid solution 4 to facilitate the plasticity and fluidity of the bioresorbable bone graft paste. The binder comprises at least one organic polymer, and the organic polymer can be synthetic organic polymer or natural organic polymer. The synthetic organic polymer can be any one or more of polyactic acid (PLA), poly(lactic-co-glycolic acid) (PLGA), poly-L-lactide (PLLA), poly-DL-lactic acid (PDLLA), and polycaprolactone (PCL), polyethylene glycol, poly(α-hydroxy ester), poly(N-isopropryl acrylamide), pluronic block copolymers, and carboxymethyl cellulose. The pluronic block copolymers consist of ethylene oxide (EO) and propylene oxide (PO), and are arranged in the structure of EOₓ-PO_{y}-EOₓ. The exact compositions of different species of pluronic block copolymers are known to those skilled in the art. The natural organic polymer can be any one or more of agarose gel, alginate, carrageenan, chitosan, collagen, fibrinogen, gelatin, hyaluronic acid, and starch. The solid solution 4 is mixed with the binder before being mixed with the liquid.

To form a paste form bioresorbable bone graft, the solid solution 4 may be mixed with a hardening agent containing metal ions before being mixed with the binder or the liquid. The solid solution 4 powder can be mixed with the hardening agent to form a powder mixture. The hardening agent containing metal compounds is used to facilitate the hardening process when the bioresorbable bone graft paste is implanted. The hardening agent may comprise one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), magnesium carbonate (MgCO₃), strontium carbonate (SrCO₃), sodium hydrogen phosphate (Na₂HPO₄) and the bioglass. The biologlass is a mixture of silica (SiO₂), sodium oxide (Na₂O), calcium oxide (CaO) and phosphorous pentoxide (P₂O₅). The specific proportion of above compounds in the bioglass is known to those skilled in the art.

A bioresorbable bone graft comprising solid solution 4 may further comprise antiosteoporotic agents other than strontium compounds. The antiosteoporotic agents may be infused in the bioresorbable bone graft and released locally with calcium ion (Ca²⁺) in the bioresorbable bone graft after implantation. The antiosteoporotic agents include therapeutic protein, therapeutic steroidal compound, bisphosphonate or other possible candidates for treating osteoporosis. The therapeutic protein can be bone morphogenetic protein (BMP), RANKL inhibiting antibody, calcitonin, parathyroid hormone or platelet-derived growth factor (PDGF). The therapeutic steroidal compound can be estrogen or selective estrogen modulator.

The method for implanting a bioresorbable bone graft comprising solid solution 4 comprises the following steps: identify the location, prepare the location, and deliver the bioresorbable bone graft to the location. X-ray computed tomography (CT), magnetic resonance imaging (MRI), ultrasonography, fluoroscopy or any other medical imaging instrument that present the image of bones in the patient can be used by the surgeon to identify the location of bone defects. The location of bone defects are the osteoporotic fracture sites or traumatized sites of the bone.

To prepare the location, the surgeon may create one or more surgical incision above or near the location. Muscle tissues, nervous tissues, epithelium or any tissues other than bone tissues above the locations may be temporarily rearranged to expose the location. The location of bone defects may be physically adjusted by drilling to form the implantation location. The implantation location for the bioresorbable bone graft includes the location of bone defects partially or entirely. The bone receiving implantation in the human body can be any one of long bone, short bone, flat bone or irregular bone. The diagnostic and surgical procedures required to identify and prepare the location are known by those skilled in the art.

The form of bioresorbable bone graft comprising solid solution 4 is relevant to the delivery of the bone graft. The bioresorbable bone graft pellets may be put into the implantation location directly. However, if the bioresorbable bone graft is to be delivered into the implantation location in the form of paste, the surgeon may need to mix the solid solution 4 with the other materials. A bioresorbable bone graft product may be provided to the surgeon, wherein the bioresorbable bone graft product comprises the materials needed to prepare a bioresorbable bone graft paste: a powder mixture comprising solid solution 4, a binder and a liquid. The powder mixture may also comprise a hardening agent to facilitate the hardening process of bioresorbable bone graft paste. The materials in the bioresorbable bone graft product are sterilized and contained separately. An instruction for mixing the materials may be included in the bioresorbable bone graft product. The surgeon need to unpack and mix the powder mixture, the binder and the liquid in the bioresorbable bone graft product to form a bioresorbable bone graft paste. The bioresorbable bone graft paste may be filled in a container before being delivered to the implantation location. The bioresorbable bone graft comprising solid solution 4 can also be delivered to the implantation location by minimally invasive surgical procedures, including but not limited to: percutaneous vertebroplasty or kyphoplasty. The size or severity of the location of bone defects is relevant to the physical space of the implantation location, and the physical space of the implantation location is positively correlated to the amount of the bioresorbable bone graft delivered.

A bioresorbable bone graft may composed of sintered strontium compounds. The sintered strontium compound can be one or more of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂). The strontium compounds are sintered at a given temperature, and the given temperature is ranged from 800° C. to 1300° C. The bioresorbable bone graft is entirely composed of above sintered strontium compounds, so that the bioresorbable bone graft may release strontium ion (Sr²⁺) directly in the implantation location. The bioresorbable bone graft also has a higher relative density of about 80% to 95% due to the sintering.

The present disclosure will now be described more specifically with reference to the following exemplary embodiments, which are provided for the purpose of demonstration rather than limitation.

### Examples

### Example 1 Bone Graft Pellets Containing a Solid Solution of Calcium Sulfate Anhydrite (CaSO₄) and Strontium Sulfate (SrSO₄)

### 1.1 Preparation of Bone Graft Pellets

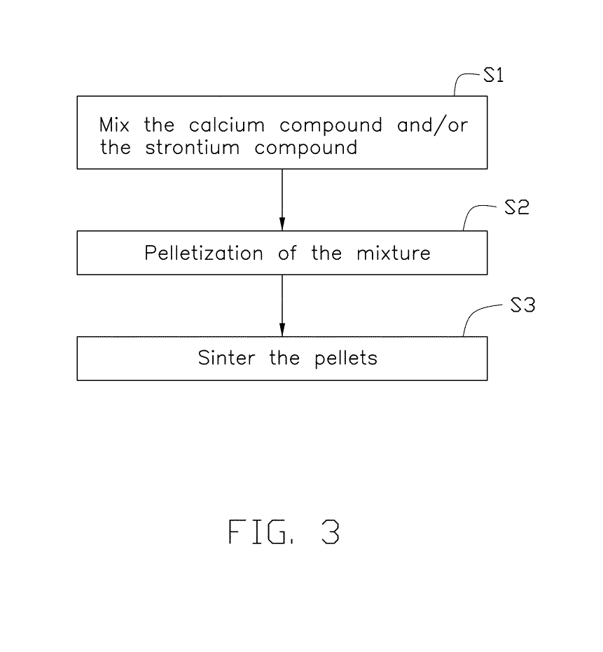

FIG. 3 describes the preparation process of bone graft pellets. In S1 of FIG. 3, a reagent grade calcium sulfate hemihydrate (CaSO₄ 0.5H₂O) powder (J. T. Baker Co., USA) and a reagent grade strontium sulfate (SrSO₄) powder (Alfa Aesar, USA) are mixed. The molar percentage for each compound of the above mixture is 7% of strontium sulfate (SrSO₄) and 93% of calcium sulfate hemihydrate. The characteristics of calcium sulfate hemihydrate (CaSO₄ 0.5H₂O) powder and strontium sulfate (SrSO₄) powder are as follows:

TABLE 1
Characteristics of calcium sulfate hemihydrate (CaSO₄•0.5H₂O)
powder and strontium sulfate (SrSO₄) powder

| Materials | Density (g/cm3) | Purity (%) | Molar mass (amu) |
|---|---|---|---|
| SrSO₄ | 3.96 | 99.9965% | 183.68 |
| CaSO₄•0.5H₂O | 2.5 | 98% | 145.15 |

The mixture of above calcium sulfate hemihydrate (CaSO₄ 0.5H₂O) powder and strontium sulfate (SrSO₄) powder is then ball-milled by zirconia balls in 99.5% ethanol solution for 4 hours. The ball-milled mixture is dried in a rotary evaporator to remove the remaining ethanol solution for 1 night. The dried ball-milled mixture is sieved through a #150 plastic mesh. In S2 of FIG. 3, a dried ball-milled mixture is consolidated into pellets under a uniaxial pressure of 25 MPa after the sieving.

In S3 of FIG. 3, the pellet-form mixture is sintered after pelletization. The sintering profile composes two steps. First, the pellet-form mixture is heated until the temperature reaches 400° C., with a temperature elevating rate of 1° C./min. The crystallization water of calcium sulfate hemihydrate (CaSO₄.5H₂O) is eliminated after the 1^{st} step and transformed into calcium sulfate anhydrite (CaSO₄). Secondly, different groups of pellet-form mixture is heated until a given temperature for each group is reached. The given temperatures for different groups of pellet-form mixture are 500° C., 600° C., 700° C., 800° C., 900° C., 1000° C., 1100° C. and 1200° C. Different groups of pellet-form mixture are heated at the above given temperatures for 1 hour.

During the sintering, the pellet-form mixtures are transforming into solid solutions. The pellet-form mixtures sintered at different temperatures are categorized according to the sintering temperatures. Sr-CS-500 is a pellet-form solid solution sintered at 500° C. Sr-CS-600 is a pellet form solid solution sintered at 600° C. Sr-CS-700 is a pellet form solid solution sintered at 700° C. Sr-CS-800 is a pellet form solid solution sintered at 800° C. Sr-CS-900 is a pellet form solid solution sintered at 900° C. Sr-CS-1000 is a pellet form solid solution sintered at 1000° C. Sr-CS-1100 is a pellet form solid solution sintered at 1100° C. Sr-CS-1200 is a pellet form solid solution sintered at 1200° C. Sr-CS-500, Sr-CS-600, Sr-CS-700, Sr-CS-800, Sr-CS-900, Sr-CS-1000, Sr-CS-1100 and Sr-CS-1200 are prepared.

A reagent grade calcium sulfate hemihydrate (CaSO₄ 0.5H₂O) powder (J. T. Baker Co., USA) is ball-milled, sieved, pelletized and sintered by the same protocol of [0097] and [0098]. Sintered calcium sulfate anhydrite (CaSO₄) pellets are categorized by the same method of [0099]. Thus, CS-500, CS-600, CS-700, CS-800, CS-900, CS-1000, CS-1100 and CS-1200 are prepared.

A reagent grade strontium sulfate (SrSO₄) powder (Alfa Aesar, USA) is ball-milled, sieved, pelletized and sintered by the same protocol of [0097] and [0098]. Sintered strontium sulfate (SrSO₄) pellets are also categorized by the same method of [0099]. Thus, Sr-500, Sr-600, Sr-700, Sr-800, Sr-900, Sr-1000, Sr-1100 and Sr-1200 are prepared.

### 1.2 Solubility Tests and Degradation Tests

### 1.2.1 Solubility Tests

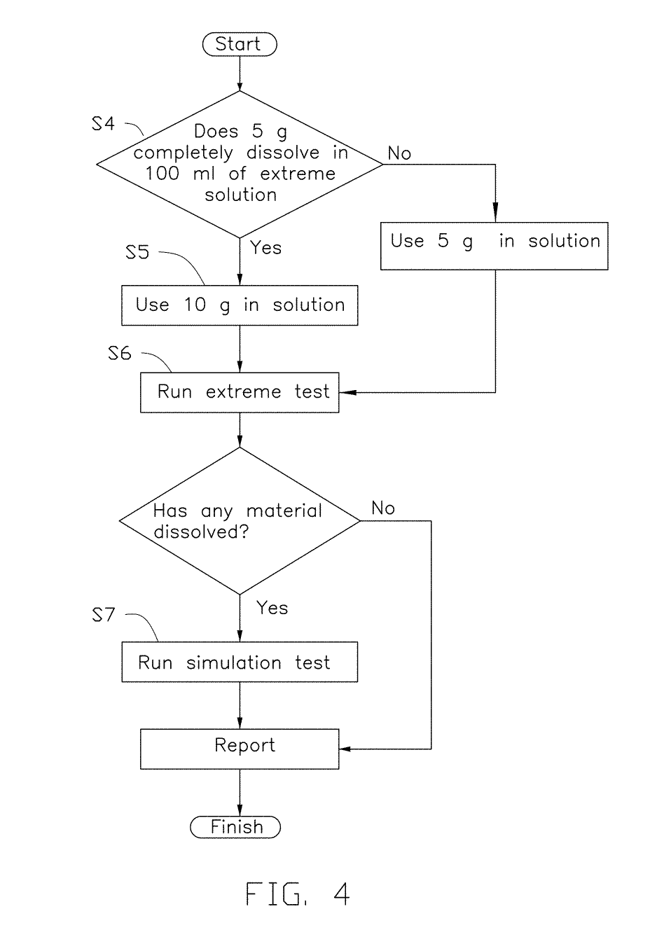

FIG. 4 illustrates the flow chart of the solubility tests of the bioresorbable bone graft pellets. The solubility tests are used to determine the dissolution behavior of a material. The flowchart of solubility tests is in accordance with ISO 10993-14. A reagent grade calcium sulfate hemihydrate (CaSO₄ 0.5H₂O) is ball-milled and sintered at 1100° C. to form CS-1100-powder. A reagent grade strontium sulfate (SrSO₄) is ball-milled and sintered at 1000° C. to form Sr-1000-powder. Calcium sulfate hemihydrate (CaSO₄ 0.5H₂O) and strontium sulfate (SrSO₄) is mixed, ball-milled and sintered at 1200° C. to form Sr-CS-1200-powder. CS-1100-powder, Sr-CS-1200-powder and Sr-1000-powder are the samples for the solubility tests and degradation tests. In S4 of FIG. 4, 5 g of CS-1100-powder, Sr-CS-1200-powder and Sr-1000-powder are added into 100 mL buffered citric acid solution. The buffered citric acid solution containing the samples are put into a water bath with a controlled temperature at 37° C., with an oscillation frequency for 12 rpm for 120 hours. A weight of 5 g of CS-1100-powder, Sr-CS-1200-powder and Sr-1000-powder are not dissolved after 120 hours. According to the results of above test and ISO10993-14, 5 g of CS-1100-powder, Sr-CS-1200-powder and Sr-1000-powder are used for an extreme test.

S5 and S6 of FIG. 4 are some of the parameters needed for an extreme test. 10 g of the samples are required in the extreme test. In S6 of FIG. 4, 5 g of CS-1100-powder, Sr-CS-1200-powder and Sr-1000-powder are added into 100 mL buffered citric acid solution. The buffered citric acid solution containing the samples are put into a water bath with a controlled temperature at 37° C., with an oscillation frequency for 12 rpm for 120 hours.

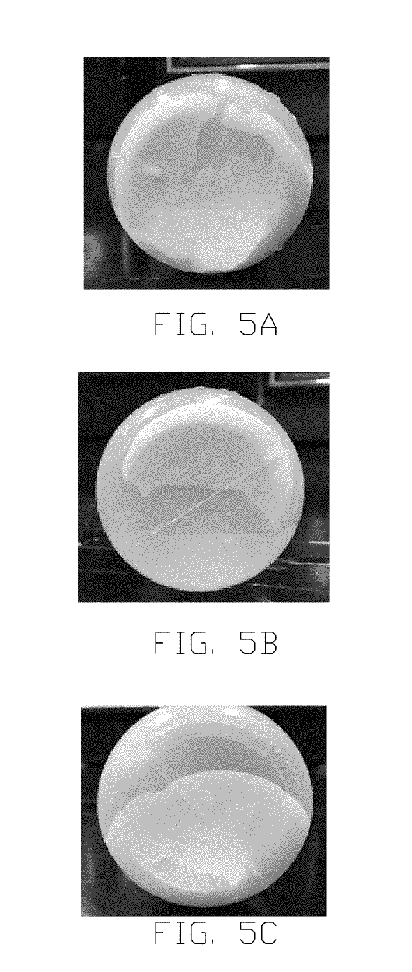

FIG. 5A, FIG. 5B and FIG. 5C are the appearances of the samples in the buffered citric acid solution after the extreme test. FIG. 5A is the remains of CS-1100-powder in the buffered citric acid solution after the extreme test. FIG. 5B is the appearance of Sr-CS-1200-powder in the buffered citric acid solution after the extreme test. FIG. 5C is the appearance of Sr-1000-powder in the buffered citric acid solution after the extreme test. The samples of S5 and S6 are not dissolved within 120 hours, therefore the buffered citric acid solutions containing the samples are filtrated by filter papers. The samples retained on the filter papers are put into a 100° C. oven overnight to be dried. The weight of dried samples are weighed, and the weight of the samples are compared with the original weight before the extreme test.

According to the result of extreme test and the flowchart in FIG. 4, a simulation test is needed to evaluate the solubility of the samples. S7 of FIG. 4 describes a simulation test. 5 g of CS-1100-powder, Sr-CS-1200-powder and Sr-1000-powder are added into 100 mL TRIS-HCl buffer solution. The TRIS-HCl buffer solution containing the samples are put into a water bath with a controlled temperature at 37° C., with an oscillation frequency for 12 rpm for 120 hours. The samples of S7 are not dissolved within 120 hours, therefore the TRIS-HCl buffer solution containing the samples are filtrated by filter papers. The samples retained on the filter papers are put into a 100° C. oven overnight to be dried. The weight of dried samples are weighed, and the weight of the samples are compared with the original weight before the simulation test. The TRIS-HCl buffer containing the samples are analyzed with inductive coupled plasma mass spectroscopy (ICP-MS: SCIEX ELAN 5000, Perkin Elmer Co., USA) to measure the release of calcium ion (Ca²⁺) and strontium ion (Sr²⁺).

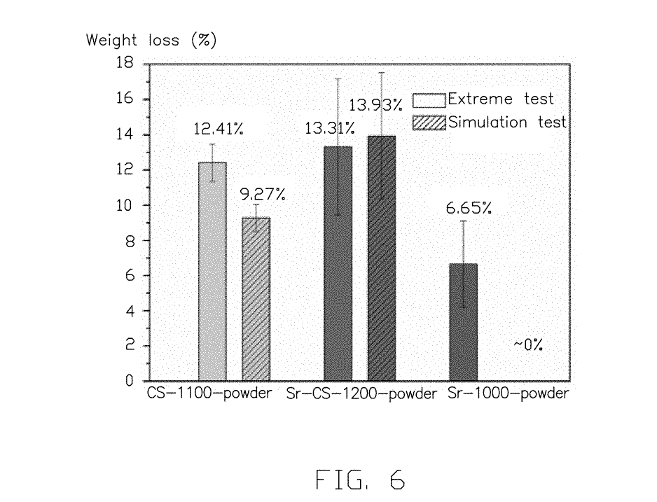

FIG. 6 illustrates the weight loss of the bioresorbable bone graft after the extreme test and the simulation test. The weight loss of CS-1100-powder in the extreme test (12.41%) is slightly higher than the weight loss in the simulation test (9.27%). The weight loss of Sr-CS-1200-powder in the extreme test (13.31%) is almost the same as the weight loss in the simulation test (13.94%). There is no weight loss of Sr-1000-powder in the simulation test, whereas the weight loss of Sr-1000-powder in the extreme test is lower than that of CS-1100-powder and Sr-CS-1200-powder. FIG. 6 demonstrates the solubility of Sr-CS-1200-powder in acidic environment provided in the extreme test is slightly higher than the solubility of CS-1100-powder and Sr-1000-powder in acidic environment. The solubility of Sr-CS-1200-powder in neutral environment provided in the simulation test is higher than the solubility of CS-1100-powder and Sr-1000-powder in neutral environment.

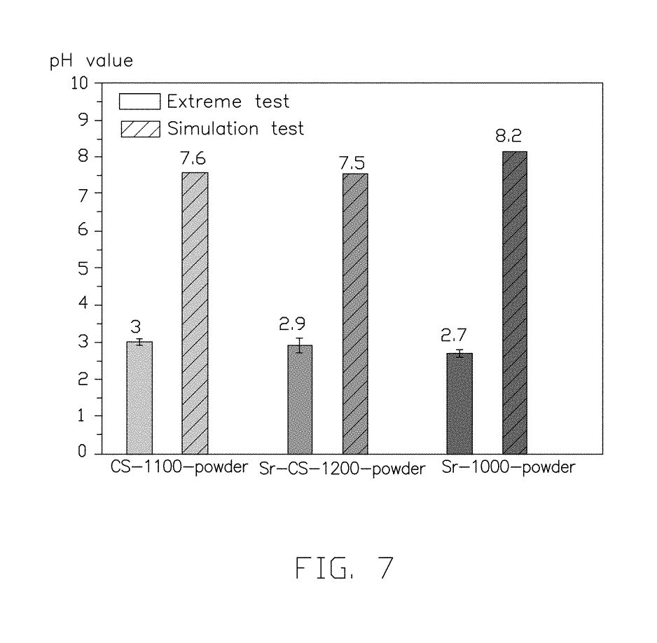

FIG. 7 illustrates the pH value of the solutions in the extreme test and the simulation test for the bioresorbable bone graft pellets. The soaking of CS-1100-powder, Sr-CS-1200-powder and Sr-1000-powder does not change the pH value of buffered citric acid solutions and TRIS-HCl buffer solutions significantly. The results of the solubility tests demonstrate the weight loss of Sr-CS-1200-powder is higher than CS-1100-powder and Sr-1000-powder, and the dissolution of Sr-CS-1200-powder does not change the pH value of surrounding environment.

Table 2 summarizes the average release rate of strontium ion (Sr²⁺) and calcium ion (Ca²⁺) released by CS-1100-powder, Sr-CS-1200-powder and Sr-1000-powder in the simulation test described in [00105] and S7 in FIG. 4. The concentration of strontium ion (Sr²⁺) released by Sr-1000-powder is around 51 ppm. Sr-1000-powder releases more strontium ion (Sr²⁺) than Sr-CS-1200-powder.

TABLE 2
The release rate of calcium ion (Ca²⁺) and strontium ion
(Sr²⁺) of CS-1100-powder, Sr-CS-1200-powder and
Sr-1000-powder after the simulation test shown in FIG. 4.

| | Average release rate | Average release rate | Weight |
|---|---|---|---|
| | of strontium ion | of calcium ion | loss |
| Material | (Sr²⁺) (ppm/day) | (Ca²⁺) (ppm/day) | (%/day) |
| CS-1100-powder | N.A. | 318.5 | 1.85% |
| Sr-CS-1200-powder | 14 | 375.5 | 2.79% |
| Sr-1000-powder | 51 | N.A. | ~0% |

### 1.2.2 Degradation Tests

A degradation test can be applied to simulate the degradation behavior of CS-1100, Sr-CS-1200 and Sr-1000 in human body. 0.25 g of CS-1100, Sr-CS-1200 and Sr-1000 are immersed in 2.5 mL of phosphate buffered saline solution. The phosphate buffered saline solution containing the samples are put into a water bath with a controlled temperature at 37° C., with an oscillation frequency for 60 rpm for 24 hours. The phosphate buffered saline solutions containing the samples are filtrated by filter papers. The phosphate buffered saline solutions containing the samples are separated with the samples, and the solutions are analyzed by ICP-MS. The samples retained on the filter papers are put into a 100° C. oven overnight to be dried. The weight of dried samples are weighed, and the samples are added in a fresh phosphate buffered saline solution for another 24 hours with oscillation and temperature-controlled water bath. The above cycle of soaking, filtering, analyzing with ICP-MS, drying and weighing is repeated 28 times.

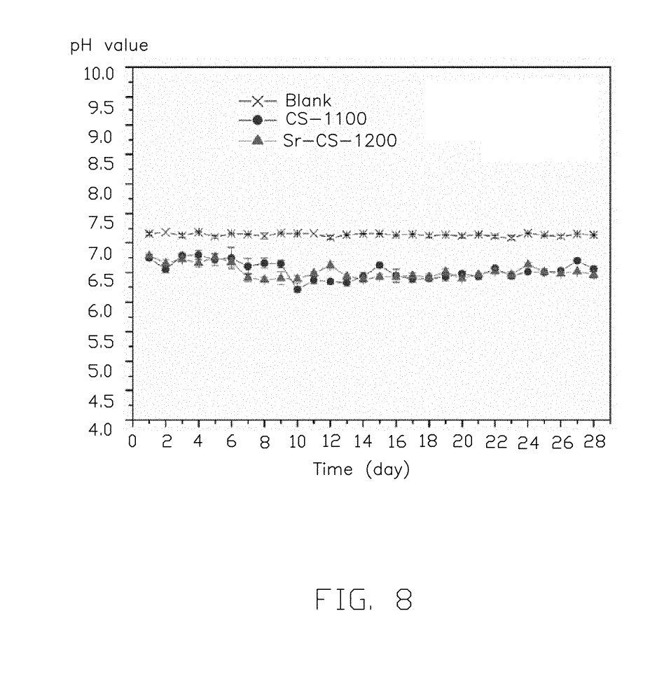

FIG. 8 illustrates the pH value of the solutions during the degradation test for CS-1100 and Sr-CS-1200. The blank in FIG. 8 is a plain phosphate buffered saline solution. After 28 days, the pH values of the phosphate buffered saline solutions containing CS-1100 or Sr-CS-1200 are slightly decreased. FIG. 8 demonstrates the degradation of CS-1100 and Sr-CS-1200 would not affect the pH of surrounding environment significantly.

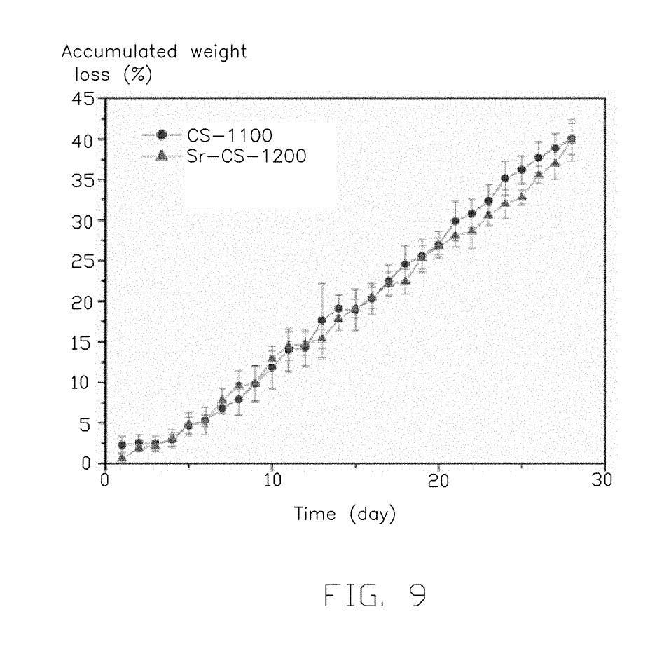

FIG. 9 illustrates the accumulated weight loss of CS-1100 and Sr-CS-1200 during the degradation test. After 28 days, the accumulated weight loss of CS-1100 is 40%, and the accumulated weight loss of Sr-CS-1200 is 39.9%. The rate of weight loss for CS-1100 is about 1.4%/day, and the rate of weight loss for Sr-CS-1200 is about 1.4%/day.

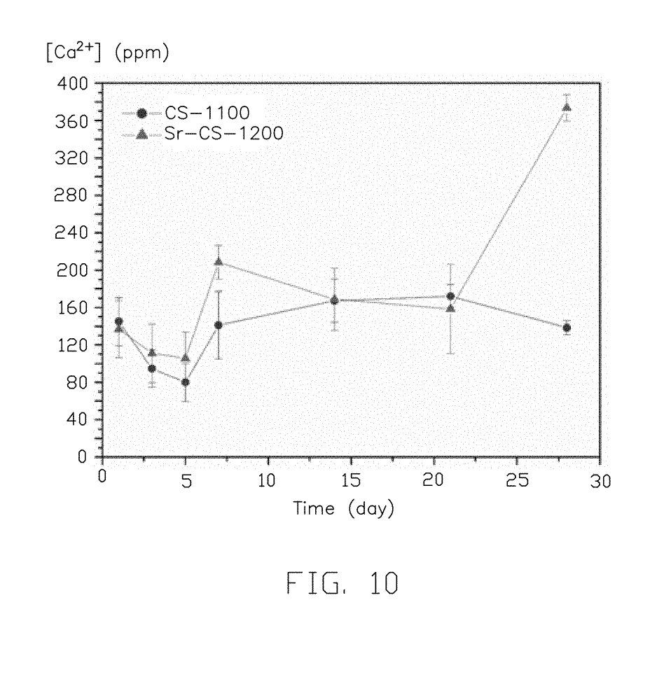

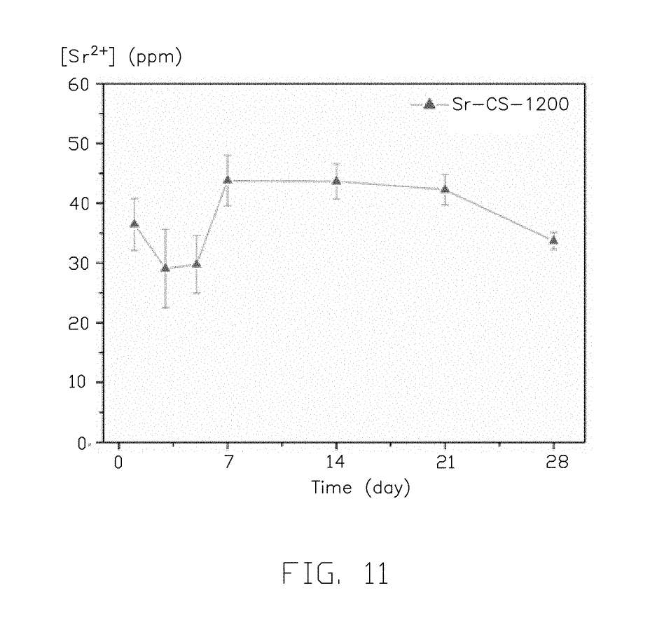

FIG. 10 and FIG. 11 are the ICP-MS result of the phosphate buffered saline solutions containing the samples from day 1 to day 28. FIG. 10 is the concentration of calcium ion (Ca²⁺) released by CS-1100 and Sr-CS-1200 during the degradation test. The concentration of calcium ion (Ca²⁺) in Sr-CS-1200 soaked solutions are slightly higher than CS-1100 soaked solutions from day 1 to day 7, and the concentration of calcium ion (Ca²⁺) is higher in Sr-CS-1200 soaked solution in day 28.

FIG. 11 is the concentration of strontium ion (Sr²⁺) released by Sr-CS-1200 during the degradation test. The concentration of strontium ion (Sr²⁺) in Sr-CS-1200 soaked solutions are around 30 ppm to 40 ppm, and the concentration of strontium ion (Sr²⁺) is around 43 ppm/day after day 7. The results of the degradation tests demonstrate the accumulated weight loss of Sr-CS-1200 in the phosphate buffered saline solution is almost the same with CS-1100, and Sr-CS-1200 releases strontium ion (Sr²⁺) when dissolved in the phosphate buffered saline solution.

### 1.3 Cytotoxicity Tests

Cytotoxicity tests are used to evaluate the potential toxicity of the samples to ex-vivo cell cultures. The preosteoblastic MC3T3-E1 cells is used in the cytotoxicity tests. The cells are fed in the α-minimum essential medium (α-MEM, Gibco, USA), which are supplemented with 10% fetal bovine serum (FBS, Gibco, USA), 100 μg/mL penicillin, 100 U/mL streptomycin, 250 ng/mL fungizone and 50 μg/mL gentamycin (Gibco, USA). The cells are cultured in an incubator with a humid atmosphere of 95% air and 5% CO₂ at 37° C.

### 1.3.1 Cytotoxicity Test on Extracts

CS-1100, Sr-CS-1200 and Sr-1000 are selected for cytotoxicity test on extracts. CS-1100, Sr-CS-1200 and Sr-1000 are immersed in the α-minimum essential medium at a ratio of 0.2 g/mL for 3 days. The sample immersed mediums are centrifuged and filtrated through a 0.22 μm filter.

The MC3T3-E1 cells are cultured in a 96-well microplate with fresh α-minimum essential medium, and each well is seeded with 10⁴ cells. After 24 hours of cell attachment, fresh α-minimum essential medium is replaced with sample immersed α-minimum essential medium. The cells are cultured in the sample immersed α-minimum essential medium for 7 days. The cells is treated by Cell Counting Kit-8 (CCK-8, Enzo Sciences, USA) to evaluate the mitochondrial activity of the cells. A microplate reader (Infinite 200 PRO, Team Co., Switzerland) is used to measure the optical absorbance at the wavelength of 450 nm of the CCK-8 treated cells.

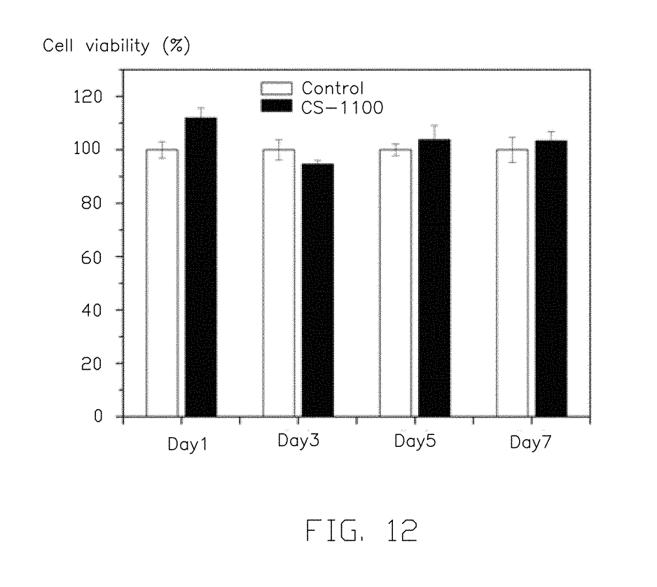

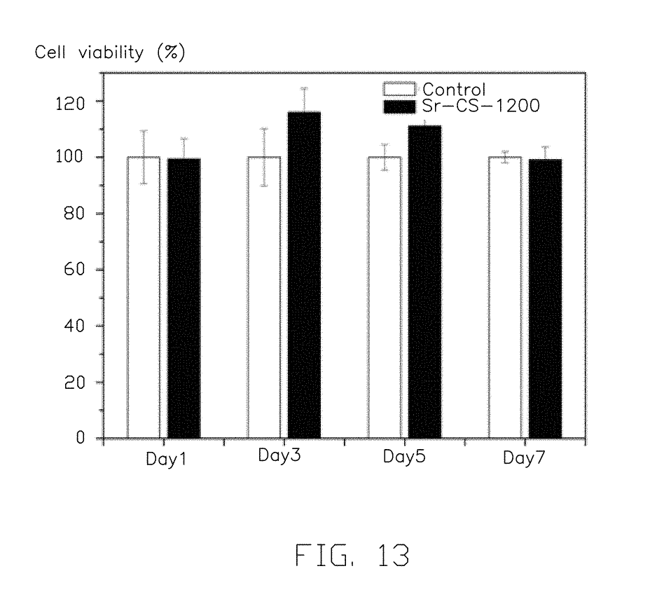

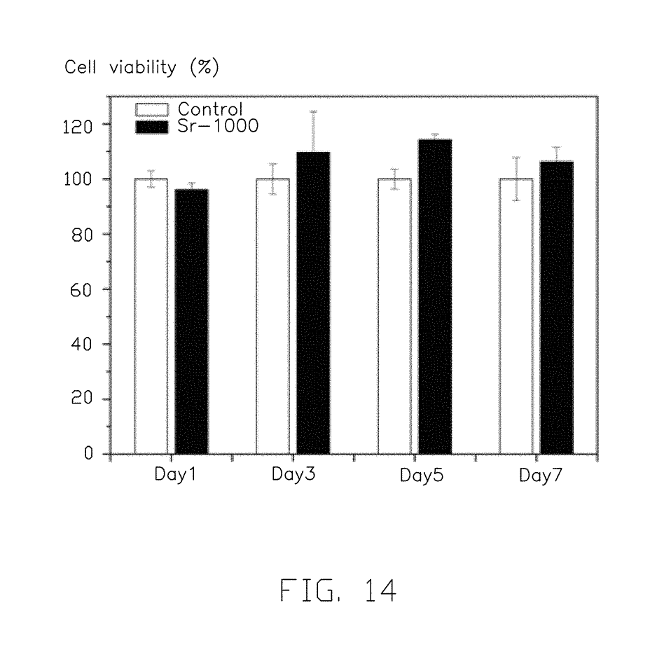

FIG. 12 illustrates the cell viability of the cytotoxicity test on extracts of CS-1100, FIG. 13 illustrates the cell viability of the cytotoxicity test on extracts of Sr-CS-1200, and FIG. 14 illustrates the cell viability of the cytotoxicity test on extracts of Sr-1000. The control group are cells with plain α-minimum essential medium. The results of cytotoxicity test on extracts of CS-1100 are 112% cell viability on day 1, 94.7% on day 3, 103.8% on day 5, and 103.4% on day 7. The results of cytotoxicity test on extracts of Sr-CS-1200 are 99.4% cell viability on day 1, 116% on day 3, 111.2% on day 5, and 99.1% on day 7. The results of cytotoxicity test on extracts of Sr-1000 are 96% on day 1, 110% on day 3, 114% on day 5, and 106% on day 7. The cell viabilities of cells immersed with CS-1100, Sr-CS-1200 and Sr-1000 are higher than the control group. The results of the cytotoxicity tests on extracts show no cytotoxicity to MCT3T3-E1 cells during the cell culture period.

### 1.3.2 Cytotoxicity Test by Direct Contact

CS-1100, Sr-CS-1200 and Sr-1000 are selected for cytotoxicity test by direct contact. Firstly, CS-1100, Sr-CS-1200 and Sr-1000 are immersed in α-minimum essential medium for 3 days. After 3 days, the α-minimum essential medium are removed and 10⁴ MCT3T3-E1 cells are put on each of the samples. The samples with the cells are then put in a 24-well microplate. Finally, fresh α-minimum essential medium are added into the samples, and the samples are cultured in an incubator with a humid atmosphere of 95% air and 5% CO₂ at 37° C. for 72 hours. After 24 hours of culture, the 400× magnification images of the cells are taken by confocal microscopy (Leica TCS SP8X, Leica Microsystems, Germany) and Cellstain double staining kit (Dojindo, Japan); the 1000× magnification images of the cells are taken by field emission scanning electronic microscopy (FE-SEM, Hitachi SU8220, Hitachi, Japan). After 72 hours of culture, the 400× magnification images of the cells are taken by confocal microscopy and Cellstain double staining kit, and the 1000× magnification images of the cells are taken by FE-SEM.

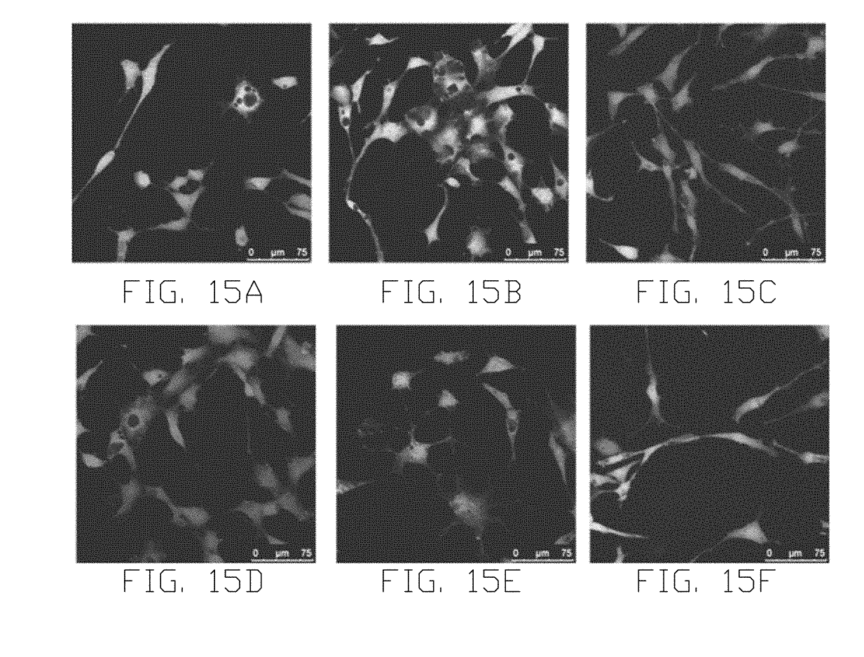

FIG. 15A and FIG. 15D are the cells' morphology under 400× magnification when cultured with CS-1100 for 24 hours. FIG. 15B and FIG. 15E are the cells' morphology under 400× magnification when cultured with Sr-CS-1200 for 24 hours. FIG. 15C and FIG. 15F are the cells' morphology under 400× magnification when cultured with Sr-1000 for 24 hours. The cells' morphologies in FIG. 15A, FIG. 15B, FIG. 15C, FIG. 15D, FIG. 15E and FIG. 15F demonstrate the cells are adhering to the surface of CS-1100, Sr-CS-1200 and Sr-1000 after 24 hours of culture. Most of the cells in FIG. 15A, FIG. 15B, FIG. 15C, FIG. 15D, FIG. 15E and FIG. 15F can be stained and observed, meaning they are alive after 24 hours of culture. Table 3 demonstrates the cell viability of MC3T3-E1 cells after 24 hour-culture with CS-1100, Sr-CS-1200 and Sr-1000.

TABLE 3
The cell viability of MC3T3-E1 cells after cultured with CS-1100,
Sr-CS-1200 and Sr-1000 for 24 hours. The control group are
cells not being put on the surface of the pellets.

| Material | Cell viability |
|---|---|
| CS-1100 | 108% |
| Sr-CS-1200 | 110% |
| Sr-1000 | 141% |
| Control | 100% |

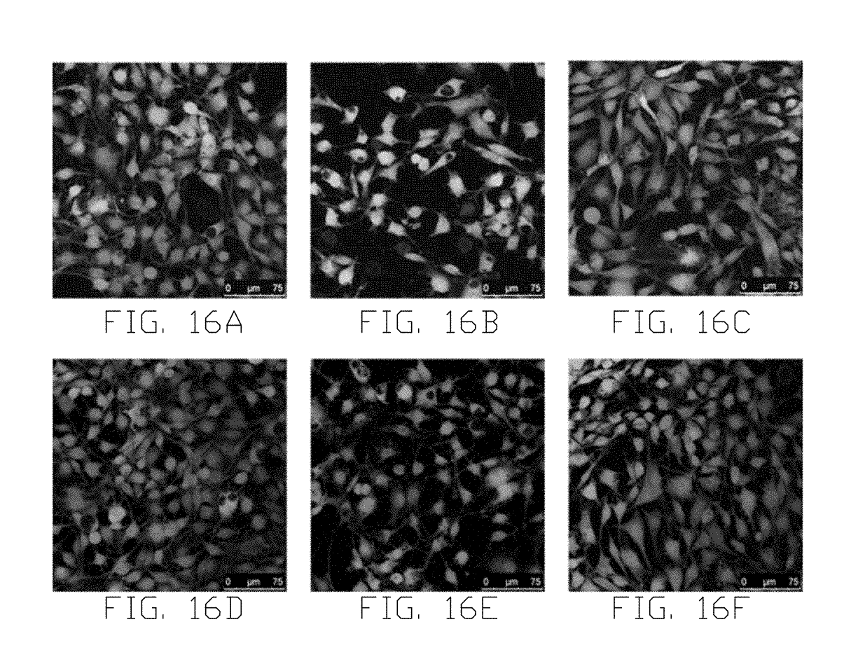

FIG. 16A and FIG. 16D are the cells' morphology under 400× magnification when cultured with CS-1100 for 72 hours. FIG. 16B and FIG. 16E are the cells' morphology under 400× magnification when cultured with Sr-CS-1200 for 72 hours. FIG. 16C and FIG. 16F are the cells' morphology under 400× magnification when cultured with Sr-1000 for 72 hours. The cells' morphologies in FIG. 16A, FIG. 16B, FIG. 16C, FIG. 16D, FIG. 16E and FIG. 16F demonstrate the cells are adhering to the surface of CS-1100, Sr-CS-1200 and Sr-1000 after 72 hours of culture. Most of the cells in FIG. 16A, FIG. 16B, FIG. 16C, FIG. 16D, FIG. 16E and FIG. 16F can be stained and observed, demonstrating the cells are alive after 72 hours of culture. When comparing with FIG. 15A, FIG. 15B, FIG. 15C, FIG. 15D, FIG. 15E and FIG. 15F, there are significantly more cells in FIG. 16A, FIG. 16B, FIG. 16C, FIG. 16D, FIG. 16E and FIG. 16F, demonstrating the cells are not only alive but also proliferating on the surface of CS-1100, Sr-CS-1200 and Sr-1000 after 72 hours of culture. Table 4 demonstrates the cells proliferate on CS-1100, Sr-CS-1200 and Sr-1000.

TABLE 4
The cell viability of MC3T3-E1 cells after cultured
with CS-1100, Sr-CS-1200 and Sr-1000 for 72 hours.

| Material | Cell viability |
|---|---|
| CS-1100 | 361% |
| Sr-CS-1200 | 275% |
| Sr-1000 | 365% |

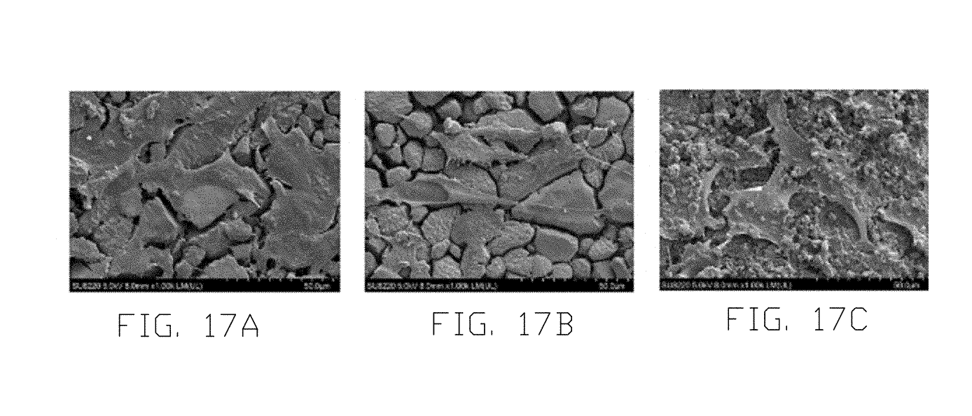

FIG. 17A is the surface image of CS-1100 under 1000× magnification when cultured with MC3T3-E1 cells for 24 hours. FIG. 17B is the surface image of Sr-CS-1200 under 1000× magnification when cultured with MC3T3-E1 cells for 24 hours. FIG. 17C is the surface image of Sr-1000 under 1000× magnification when cultured with MC3T3-E1 cells for 24 hours. Extended cytoplasm of the cells can be observed on FIG. 17A, FIG. 17B and FIG. 17C, this demonstrates the cells are adhering to the surface of CS-1100, Sr-CS-1200 and Sr-1000 after 24 hours of culture.

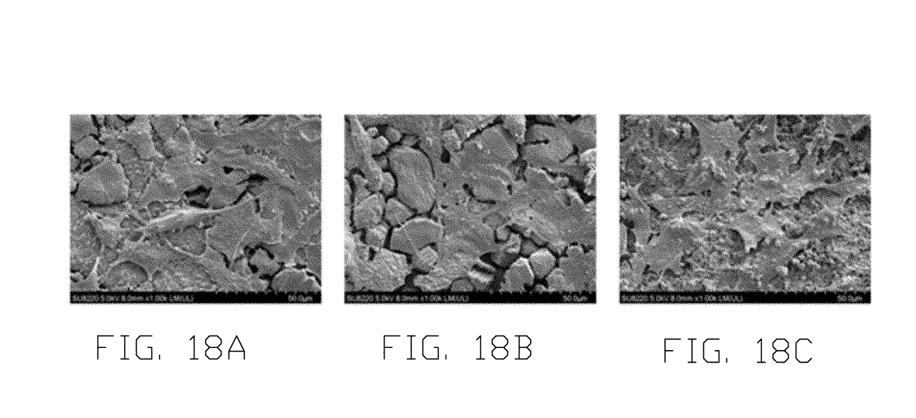

FIG. 18A is the surface image of CS-1100 under 1000× magnification when cultured with MC3T3-E1 cells for 72 hours. FIG. 18B is the surface image of Sr-CS-1200 under 1000× magnification when cultured with MC3T3-E1 cells for 72 hours. FIG. 18C is the surface image of Sr-1000 under 1000× magnification when cultured with MC3T3-E1 cells for 72 hours. Extended cytoplasm of the cells can be observed on FIG. 18A, FIG. 18B and FIG. 18C, this demonstrates the cells are adhering to the surface of CS-1100, Sr-CS-1200 and Sr-1000 after 72 hours of culture. The results of cytotoxicity tests by direct contact demonstrate MC3T3-E1 cells proliferate and adhere on CS-1100, Sr-CS-1200 and Sr-1000 after 24 hours and 72 hours.

### 1.4 Density Measurements

The densities of sintered calcium sulfate anhydrite (CaSO₄) pellets, strontium-calcium (Sr—Ca) solid solution pellets and sintered strontium sulfate (SrSO₄) pellets are calculated from their weight and volume. The densities of sintered pellets are compared with the theoretical density of each compound to calculate the relative densities. The relative density is generated by dividing the measured density of a material by the theoretical density of the same material. The theoretical density of strontium sulfate (SrSO₄) is 3.96 g/cm³, and the theoretical density of calcium sulfate anhydrite (CaSO₄) is 2.9 g/cm³. The theoretical density of strontium-calcium (Sr—Ca) solid solution can be calculated from the weight percentage of the solid solution. The 7 molar percent of strontium sulfate (SrSO₄) is 10 weight percent in the solid solution. The 93 molar percent of calcium sulfate anhydrite (CaSO₄) is 90 weight percent of calcium sulfate anhydrite (CaSO₄) after sintering. Therefore, the theoretical density for the strontium-calcium (Sr—Ca) solid solution is 10% of the theoretical density of strontium sulfate (SrSO₄) plus 90% of the theoretical density of calcium sulfate anhydrite (CaSO₄), and the theoretical density of strontium-calcium (Sr—Ca) solid solution is 3.006 g/cm³.

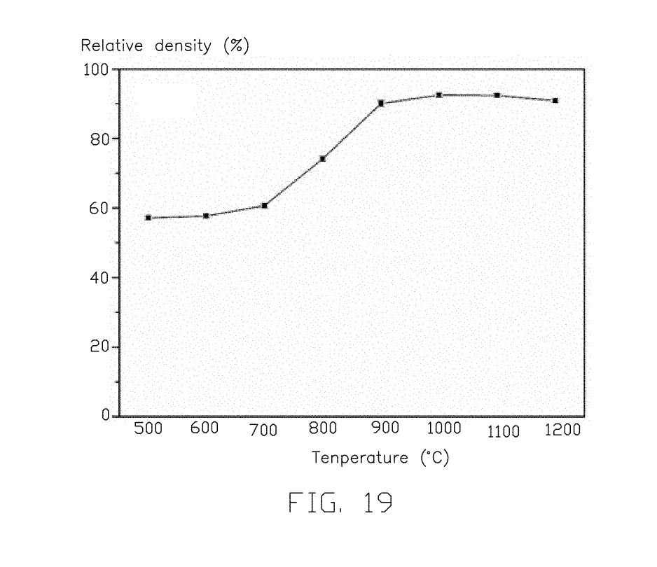

The densities of CS-500, CS-600, CS-700, CS-800, CS-900, CS-1000, CS-1100 and CS-1200 are divided by the theoretical density of calcium sulfate anhydrite (CaSO₄) respectively to generate the relative densities. FIG. 19 illustrates the relative densities of CS-500, CS-600, CS-700, CS-800, CS-900, CS-1000, CS-1100 and CS-1200. FIG. 19 shows that when the sintering temperature reaches 900° C., the relative densities of the sintered calcium sulfate anhydrite (CaSO₄) pellets are higher than 90%. The relative density of CS-1000 is 93% and CS-1100 is 92%, both exhibit higher densities than other sintered pellets.

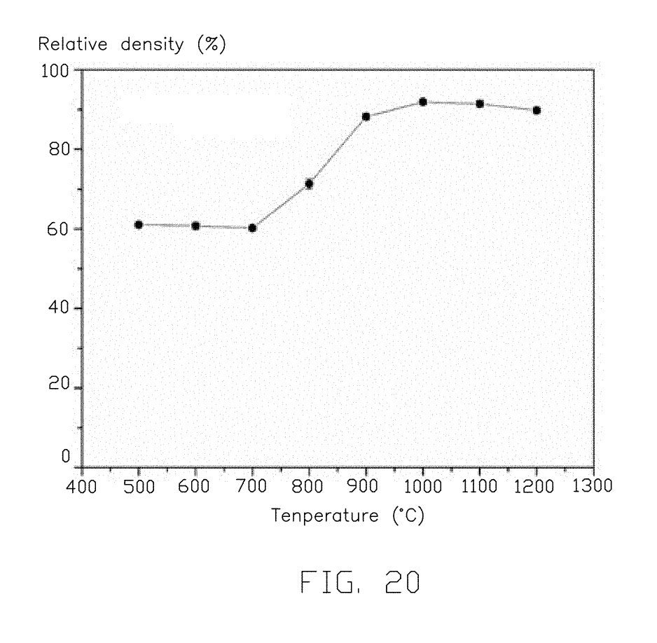

The densities of Sr-CS-500, Sr-CS-600, Sr-CS-700, Sr-CS-800, Sr-CS-900, Sr-CS-1000, Sr-CS-1100 and Sr-CS-1200 are divided by the theoretical density of strontium-calcium (Sr—Ca) solid solution respectively to generate the relative densities. FIG. 20 illustrates the relative densities of Sr-CS-500, Sr-CS-600, Sr-CS-700, Sr-CS-800, Sr-CS-900, Sr-CS-1000, Sr-CS-1100 and Sr-CS-1200. FIG. 20 shows that the relative density increases significantly between Sr-CS-700 and Sr-CS-900. The relative density of Sr-CS-700 is 60.2%, and the relative density of Sr-CS-900 is 88.2%. The relative density of Sr-CS-1000 is 91.1% and Sr-CS-1100 is 91.4%.

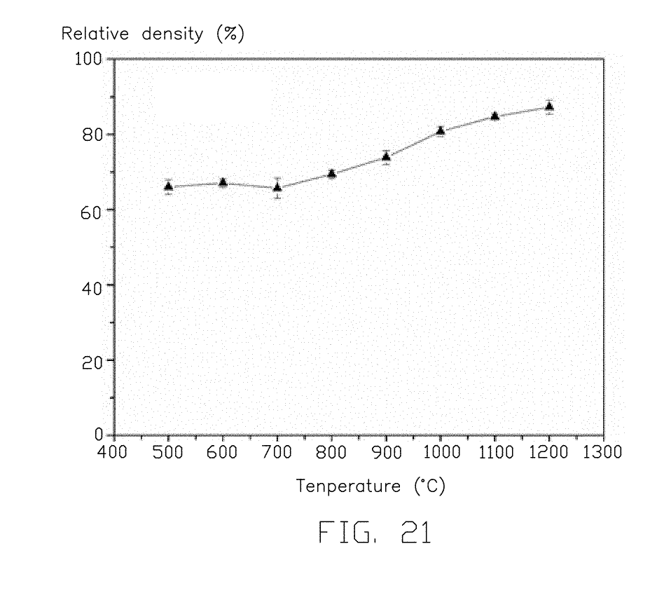

The densities of Sr-500, Sr-600, Sr-700, Sr-800, Sr-900, Sr-1000, Sr-1100 and Sr-1200 are divided by the theoretical density of strontium sulfate (SrSO₄) to generate the relative densities. FIG. 21 illustrates the relative densities of Sr-500, Sr-600, Sr-700, Sr-800, Sr-900, Sr-1000, Sr-1100 and Sr-1200. FIG. 21 shows that the relative density increases with the sintering temperature. Sr-1200 exhibits the highest relative density of 87.2%. The results of the density measurements generally demonstrate the relative density increases with the sintering temperature among sintered calcium sulfate anhydrite (CaSO₄) pellets, strontium-calcium solid solution pellets and strontium sulfate (SrSO₄) pellets.

### 1.5 X-Ray Diffraction Patterns

Sintered calcium sulfate anhydrite (CaSO₄) pellets, strontium-calcium (Sr—Ca) solid solution pellets and sintered strontium sulfate (SrSO₄) pellets are analyzed by X-ray diffraction (D2 PHASER, Bruker Co., USA) at 30 kV and 10 mA at scanning rate of 3 degree 20/min.

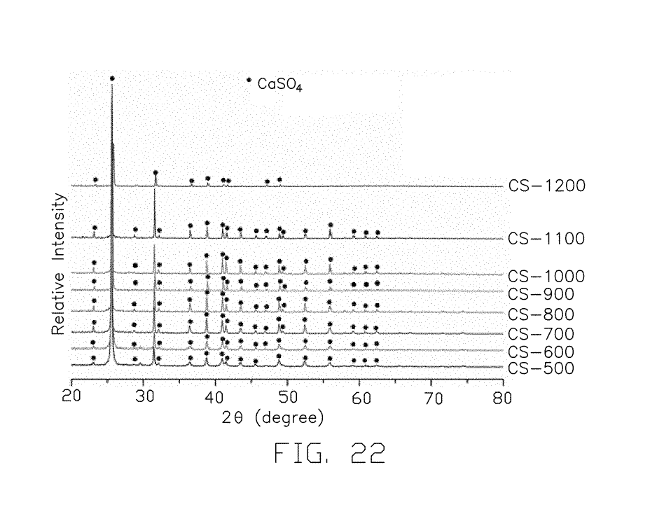

FIG. 22 is the X-ray diffraction patterns of CS-500, CS-600, CS-700, CS-800, CS-900, CS-1000, CS-1100 and CS-1200. FIG. 22 shows a single phase in above sintered calcium sulfate anhydrite (CaSO₄) pellets.

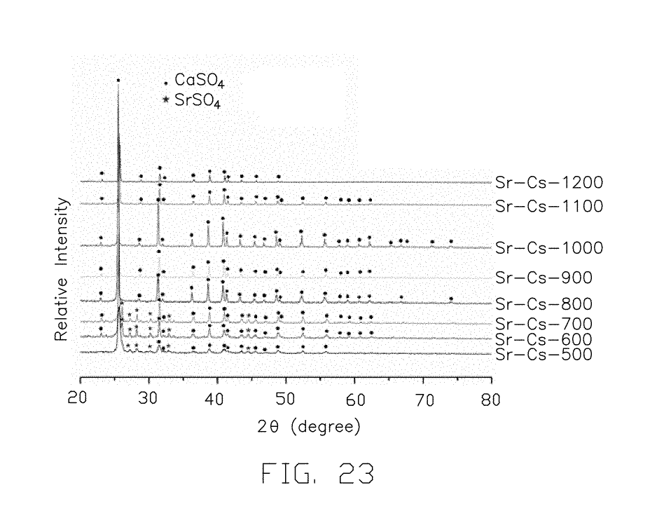

FIG. 23 is the X-ray diffraction patterns of Sr-CS-500, Sr-CS-600, Sr-CS-700, Sr-CS-800, Sr-CS-900, Sr-CS-1000, Sr-CS-1100 and Sr-CS-1200. The host in above strontium-calcium solid solution is calcium sulfate anhydrite (CaSO₄), therefore the X-ray diffraction pattern is similar to FIG. 22. FIG. 23 also shows that there is no strontium sulfate (SrSO₄) phase after the sintering temperature reaches 800° C.

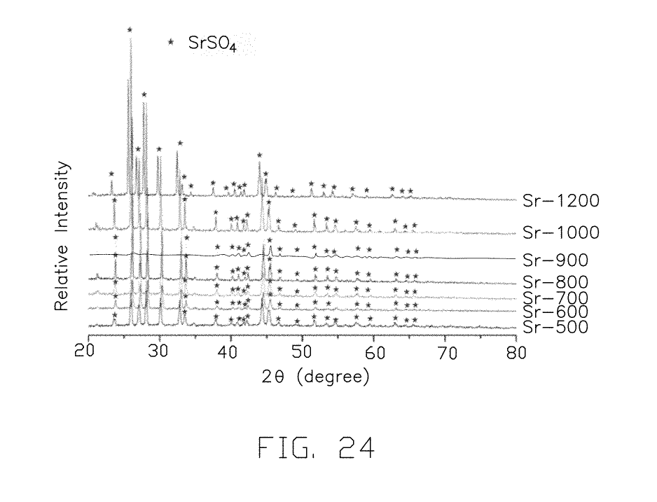

FIG. 24 is the X-ray diffraction patterns of Sr-500, Sr-600, Sr-700, Sr-800, Sr-900, Sr-1000, Sr-1100 and Sr-1200. FIG. 24 shows a single phase in above sintered strontium sulfate (SrSO₄) pellets.

### 1.6 Surface Imaging by Scanning Electronic Microscopy

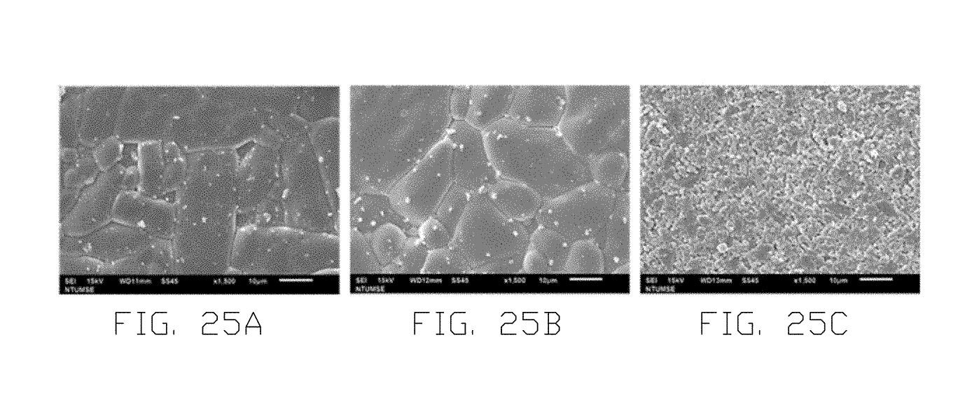

FIG. 25A, FIG. 25B and FIG. 25C are the surface of the bioresorbable bone graft pellets comprising a solid solution under 1000× magnification by scanning electronic microscopy (JSM6510, JEOL Co., Japan).

FIG. 25A is the microscopic image of CS-1100 under 1000× magnification by scanning electronic microscopy. The pores are located at the grain boundaries of calcium sulfate anhydrite (CaSO₄).

FIG. 25B is the microscopic image of Sr-CS-1200 under 1000× magnification by scanning electronic microscopy. The grains of Sr-CS-1200 closely resemble the grain of CS-1100 in FIG. 25A, because the host of Sr-CS-1200 is calcium sulfate anhydrite (CaSO₄).

FIG. 25C is the microscopic image of Sr-1000 under 1000× magnification by scanning electronic microscopy. The grain size of Sr-CS-1200 in FIG. 25B is significantly larger than the grain size of Sr-1000 in FIG. 25C. The results of surface imaging by scanning electronic microscopy show the grains of Sr-CS-1200 closely resembles the grains of CS-1100.

### 1.7 Implantation in Rat Calvarial Bone

18-week-old male Sprague-Dawley rats with body weight of 500 to 530 g are used to conduct the implantation of bone graft pellets in rat calvarial bone. The rats are anesthetized with 1-4% Isoflurane. Then, 2 defects of 5 mm in diameter are created in the central area of one rat calvarial bone by a trephine bur (ACE Surgical Supply Co., Inc.) as the implantation location for bone graft pellets. The dura mater of the rats are avoided to be perforated. Saline and antibiotic are irrigated to prepare the calvarial defect. Sr-CS-1200 and Sr-1000 are implanted in the 5 mm-defects on the rat calvarial bone.

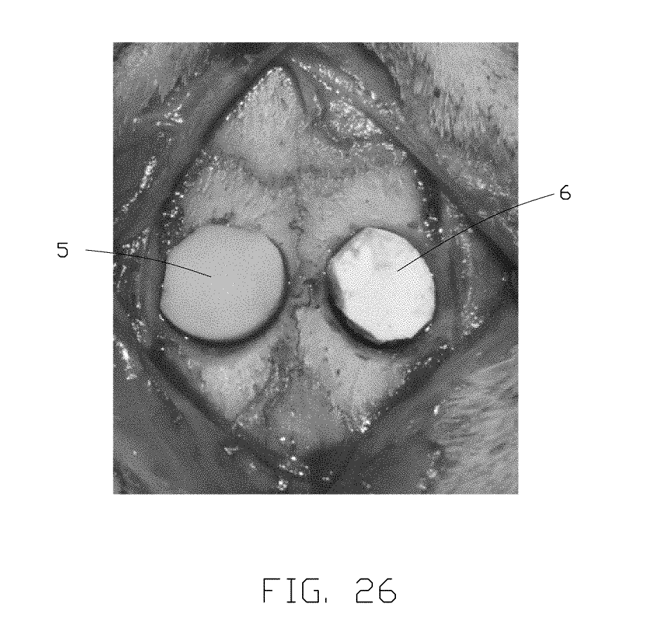

FIG. 26 is the appearance of rat calvarial bone when implanted with Sr-CS-1200 and Sr-1000 prepared in 1.1. Pellet 5 is the Sr-CS-1200 and pellet 6 is the Sr-1000. After the implantation of bone graft pellets, the muscle and soft tissue are sutured with 3-0 DEXON. The rats are euthanized with carbon dioxide (CO₂) after 3 and 6 months.

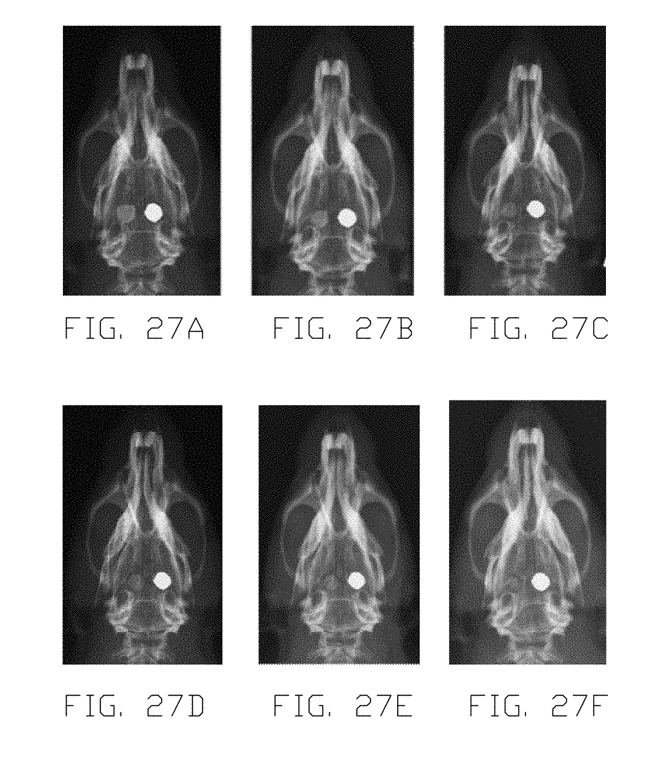

The rat calvarial defects implanted with bone graft pellets are scanned by X-ray (KXO-50R, TOSHIBA, Japan) at voltage of 45 kV and current of 100 mA. FIG. 27A, FIG. 27B, FIG. 27C, FIG. 27D, FIG. 27E and FIG. 27F are the X-ray images of rat calvarial defects implanted with Sr-CS-1200 and Sr-1000 after 2 weeks, 8 weeks, 12 weeks, 16 weeks, 20 weeks and 24 weeks, respectively. There are no distinctive bone regeneration after 2 weeks, 8 weeks, 12 weeks and 20 weeks, as exemplified in FIG. 27A, FIG. 27B, FIG. 27C, FIG. 27D and FIG. 27E. FIG. 27F shows slightly blurred edges between the rat calvarial defects and implanted Sr-CS-1200 and Sr-1000. The results of Sr-CS-1200 and Sr-1000 implantation in rat calvarial bone show bone regeneration after 24 weeks.

### Example 2 Bioresorbable Bone Graft Paste Containing the Solid Solution of Example 1

### 2.1 Preparation of Bioresorbable Bone Graft Paste

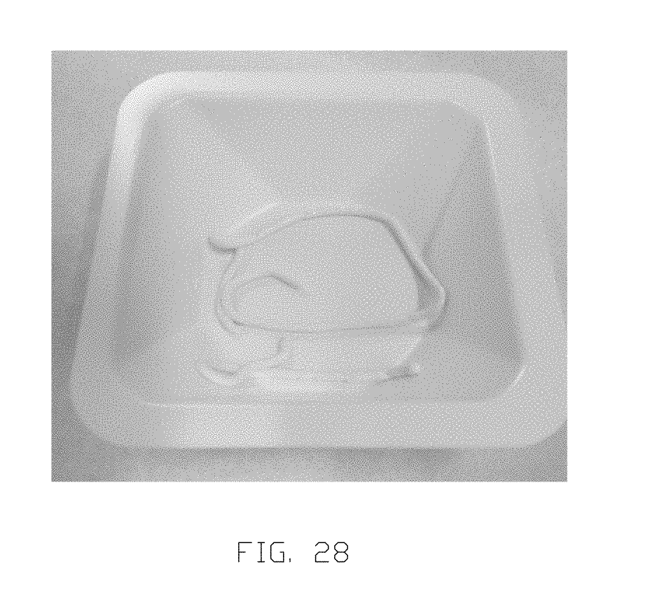

FIG. 28 shows a bioresorbable bone graft containing Sr-CS-1100 in the form of paste. Sr-CS-1100 prepared in 1.1 is finely grinded in an agate mortar to produce a powder form of Sr-CS-1100. 1.8 g of the powder form of Sr-CS-1100 is then mixed with 1.2 g calcium sulfate hemihydrate (CaSO₄ 0.5H₂O) powder and 0.03 g of food grade carboxymethyl cellulose powder for 4 hours to produce a mixture, wherein the calcium sulfate hemihydrate (CaSO₄ 0.5H₂O) powder is the hardening agent, and carboxymethyl cellulose powder is the binder. The mixture is blended with 1.5 mL 0.1 M sodium hydrogen phosphate (Na₂HPO₄) solution to form a bioresorbable bone graft paste, as exemplified in FIG. 28. The mixture can also be blended with 1.5 mL 0.5 M sodium hydrogen phosphate (Na₂HPO₄) to form a bioresorbable bone graft paste.

### 2.2 Hardening Test

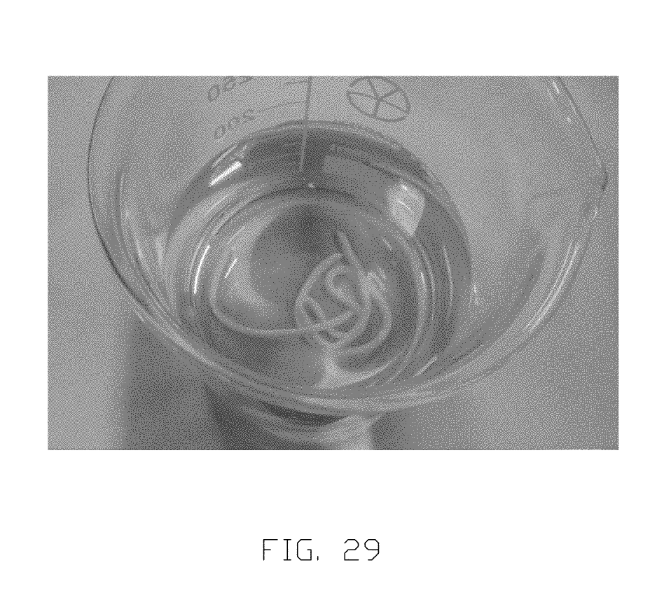

The bioresorbable bone graft paste prepared in 2.1 containing 0.1 M sodium hydrogen phosphate (Na₂HPO₄) is filled in a syringe. The syringe then injects the bone graft paste into water. FIG. 29 shows the bone graft paste does not scattered in the water after 20 minutes, thereby the bone graft paste can be implanted in the human body without disintegrate prematurely.

It will be understood that the above description of embodiments is given by way of example only and that various modifications can be made by those with ordinary skill in the art. The above specification, examples and data provide a complete description of the structure and use of exemplary embodiments. Although various embodiments have been described above with a certain degree of particularity, or with reference to one or more individual embodiments, those with ordinary skill in the art could make numerous alterations to the disclosed embodiments without departing from the spirit or scope of this disclosure.

### STATEMENTS OF THE DISCLOSURE

Statement 1: A solid solution for use in bone regeneration, comprising: at least one divalent cation, wherein the divalent cation is calcium ion (Ca²⁺) and/or strontium ion (Sr²⁺), at least one anion, and the at least one anion comprises one or more of sulfate (SO₄²⁻), phosphate (PO₄²⁻), carbonate (CO₃²⁻), and silicate (SiO₃²⁻); wherein the relative density of the solid solution is about 65% to 100%.

Statement 2: The solid solution according to Statement 1, wherein the solid solution further comprising at least one additional divalent cation, the additional divalent cation comprises one or more of magnesium ion (Mg²⁺), barium ion (Ba²⁺), and zinc ion (Zn²⁺).

Statement 3: The solid solution according to any one of Statement 1 or 2, wherein the relative density of the solid solution is about 80% to 95%.

Statement 4: The solid solution according to any one of Statement 1 to 3, wherein the molar ratio of the cation to the anion is 1 to 1.5.

Statement 5: The solid solution according to any one of Statement 1 to 4, wherein the divalent cations are calcium ion (Ca²⁺) and strontium ion (Sr²⁺), and the molar ratio of calcium ion (Ca²⁺) and strontium ion (Sr²⁺) is 1 to 33.

Statement 6: The solid solution according to Statement 5, wherein the molar ratio of calcium ion (Ca²⁺) and strontium ion (Sr²⁺) is 6 to 20.

Statement 7: The solid solution according to any one of Statement 1 to 6, wherein the solid solution is in the form of pellets.

Statement 8: A bioresorbable bone graft product for use in bioresorbable bone graft implantation, comprising: a liquid, the liquid is derived from non-biological origin; a binder, the binder is a synthetic organic polymer or a natural organic polymer; a powder mixture, and the powder mixture comprises a solid solution powder comprises at least one divalent cation and at least one anion, wherein the at least one divalent cation is calcium ion (Ca²⁺) and/or strontium ion (Sr²⁺), and the at least one anion comprises one or more of sulfate (SO₄²⁻), phosphate (PO₄²⁻), carbonate (CO₃²⁻), and silicate (SiO₃²⁻); wherein the relative density of the solid solution powder is about 65% to 100%.

Statement 9: The bioresorbable bone graft product according to Statement 8, wherein the solid solution powder further comprising at least one additional divalent cation, the additional divalent cation comprises one or more of magnesium ion (Mg²⁺), barium ion (Ba²⁺), and zinc ion (Zn²⁺).

Statement 10: The bioresorbable bone graft product according to any one of Statement 8 or 9, wherein the relative density of the solid solution powder is about 80% to 95%.

Statement 11: The bioresorbable bone graft product according to any one of Statement 8 to 10, wherein the molar ratio of the cation to the anion is 1 to 1.5 in the solid solution powder.

Statement 12: The bioresorbable bone graft product according to any one of Statement 8 to 11, wherein the divalent cations are calcium ion (Ca²⁺) and strontium ion (Sr²⁺), and the molar ratio of calcium ion (Ca²⁺) and strontium ion (Sr²⁺) is 1 to 33 in the solid solution powder.

Statement 13: The bioresorbable bone graft product according to Statement 12, wherein the molar ratio of calcium ion (Ca²⁺) and strontium ion (Sr²⁺) is 6 to 20 in the solid solution powder.

Statement 14: The bioresorbable bone graft product according to any one of Statement 8 to 13, wherein the liquid derived from non-biological origin comprises one or more of Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid.

Statement 15: The bioresorbable bone graft product according to any one of Statement 8 to 14, wherein the synthetic organic polymer comprises one or more of polyactic acid (PLA), poly(lactic-co-glycolic acid) (PLGA), poly-L-lactide (PLLA), poly-DL-lactic acid (PDLLA), polycaprolactone (PCL), polyethylene glycol, poly(α-hydroxy ester), poly(N-isopropyl acrylamide), pluronic block copolymers, and carboxymethyl cellulose.

Statement 16: The bioresorbable bone graft product according to any one of Statement 8 to 15, wherein the natural organic polymer comprises one or more of agarose gel, alginate, carrageenan, chitosan, collagen, fibrinogen, gelatin, hyaluronic acid, and starch.

Statement 17: The bioresorbable bone graft product according to any one of Statement 8 to 16, wherein the powder mixture further comprising a hardening agent, the hardening agent comprises one or more of calcium sulfate anhydrite (CaSO₄), calcium sulfate hemihydrate (CaSO₄ 0.5H₂O), calcium sulfate dihydrate (CaSO₄, 2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₈(PO₄)₃(OH), calcium carbonate (CaCO₃), magnesium carbonate (MgCO₃), strontium carbonate (SrCO₃), sodium hydrogen phosphate (Na₂HPO₄) and the bioglass.

Statement 18: A method for preparing and implanting the bioresorbable bone graft paste, comprising a) mix the powder mixture, the binder and the liquid according to any one of Statement 8 to 18 to form a bioresorbable bone graft paste; b) deliver the bioresorbable bone graft paste to one or more implantation locations.

Statement 19: A solid solution for use in bone regeneration, comprising: two divalent cations, wherein a first divalent cation is calcium ion (Ca²⁺) and a second divalent cation is selected from the group consisting of magnesium ion (Mg²⁺), strontium ion (Sr²⁺), barium ion (Ba²⁺), and zinc ion (Zn²⁺); at least one anion, and the at least one anion comprises one or more of sulfate (SO₄²⁻), phosphate (PO₄²⁻), carbonate (CO₃²⁻), and silicate (SiO₃²⁻); wherein the relative density of the solid solution is about 65% to 100%.

Statement 20: The solid solution according to Statement 19, wherein the relative density of the solid solution is about 80% to 95%.

Statement 21: The solid solution according to any one of Statement 19 or 20, wherein the molar ratio of the cation to the anion is 1 to 1.5.

Statement 22: The solid solution according to any one of Statement 19 to 21, wherein the molar ratio of the first divalent cation and the second divalent cation is 1 to 33.

Statement 23: The solid solution according to Statement 22, wherein the molar ratio of the first divalent cation and the second divalent cation is 6 to 20.

Statement 24: The solid solution according to any one of Statement 19 to 22, wherein the solid solution is in the form of pellets.

Statement 25: A bioresorbable bone graft product for use in bioresorbable bone graft implantation, comprising: a liquid, the liquid is derived from non-biological origins; a binder, the binder is a synthetic organic polymer or a natural organic polymer; a powder mixture, and the powder mixture comprises a powder formed from the solid solution according to any one of Statement 19 to 24.

Statement 26: The bioresorbable bone graft product according to Statement 25, wherein the liquid derived from non-biological origin comprises one or more of Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid.

Statement 27: The bioresorbable bone graft product according to any one of Statement 25 or 26, wherein the synthetic organic polymer comprises one or more of polyactic acid (PLA), poly(lactic-co-glycolic acid) (PLGA), poly-L-lactide (PLLA), poly-DL-lactic acid (PDLLA), polycaprolactone (PCL), polyethylene glycol, poly(α-hydroxy ester), poly(N-isopropyl acrylamide), pluronic block copolymers, and carboxymethyl cellulose.

Statement 28: The bioresorbable bone graft product according to any one of Statement 25 to 27, wherein the natural organic polymer comprises one or more of agarose gel, alginate, carrageenan, chitosan, collagen, fibrinogen, gelatin, hyaluronic acid, and starch.

Statement 29: The bioresorbable bone graft product according to any one of Statement 25 to 28, wherein the powder mixture further comprising a hardening agent, the hardening agent comprises one or more of calcium sulfate anhydrite (CaSO₄), calcium sulfate hemihydrate (CaSO₄ 0.5H₂O), calcium sulfate dihydrate (CaSO₄, 2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₈(PO₄)₃(OH), calcium carbonate (CaCO₃), magnesium carbonate (MgCO₃), strontium carbonate (SrCO₃), sodium hydrogen phosphate (Na₂HPO₄) and the bioglass.

Statement 30: A method for preparing and implanting the bioresorbable bone graft paste, comprising a) mix the powder mixture, the binder and the liquid according to any one of Statement 25 to 29 to form a bioresorbable bone graft paste; b) deliver the bioresorbable bone graft paste to one or more implantation locations.

Statement 31: A solid solution for use in bone regeneration, comprising: two divalent cations, wherein a first divalent cation is strontium ion (Sr²⁺) and a second divalent cation is selected from the group consisting of magnesium ion (Mg²⁺), barium ion (Ba²⁺), and zinc ion (Zn²⁺); at least one anion, and the at least one anion comprises one or more of sulfate (SO₄²⁻), phosphate (PO₄²⁻), carbonate (CO₃²⁻), and silicate (SiO₃²⁻); wherein the relative density of the solid solution is about 65% to 100%.

Statement 32: The solid solution according to Statement 31, wherein the relative density of the solid solution is about 80% to 95%.

Statement 33: The solid solution according to any one of Statement 31 or 32, wherein the molar ratio of the cation to the anion is 1 to 1.5.

Statement 34: The solid solution according to any one of Statement 31 to 33, wherein the molar ratio of the first divalent cation and the second divalent cation is 1 to 33.

Statement 35: The solid solution according to Statement 34, wherein the molar ratio of the first divalent cation and the second divalent cation is 6 to 20.

Statement 36: The solid solution according to any one of Statement 31 to 35, wherein the solid solution is in the form of pellets.

Statement 37: A bioresorbable bone graft product for use in bioresorbable bone graft implantation, comprising: a liquid, the liquid is derived from non-biological origins; a binder, the binder is a synthetic organic polymer or a natural organic polymer; a powder mixture, and the powder mixture comprises a powder formed from the solid solution according to any one of Statement 31 to 36.

Statement 38: The bioresorbable bone graft product according to Statement 37, wherein the liquid derived from non-biological origin comprises one or more of Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid.

Statement 39: The bioresorbable bone graft product according to any one of Statement 37 or 38, wherein the synthetic organic polymer comprises one or more of polyactic acid (PLA), poly(lactic-co-glycolic acid) (PLGA), poly-L-lactide (PLLA), poly-DL-lactic acid (PDLLA), polycaprolactone (PCL), polyethylene glycol, poly(α-hydroxy ester), poly(N-isopropyl acrylamide), pluronic block copolymers, and carboxymethyl cellulose.

Statement 40: The bioresorbable bone graft product according to any one of Statement 37 to 39, wherein the natural organic polymer comprises one or more of agarose gel, alginate, carrageenan, chitosan, collagen, fibrinogen, gelatin, hyaluronic acid, and starch.

Statement 41: The bioresorbable bone graft product according to any one of Statement 37 to 40, wherein the powder mixture further comprising a hardening agent, the hardening agent comprises one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₈(PO₄)₃(OH), calcium carbonate (CaCO₃), magnesium carbonate (MgCO₃), strontium carbonate (SrCO₃), sodium hydrogen phosphate (Na₂HPO₄) and the bioglass.

Statement 42: A method for preparing and implanting the bioresorbable bone graft paste, comprising a) mix the powder mixture, the binder and the liquid according to any one of Statement 37 to 41 to form a bioresorbable bone graft paste; b) deliver the bioresorbable bone graft paste to one or more implantation locations.

Statement 43: A bioresorbable bone graft, the bioresorbable bone graft is a strontium compound, the strontium compound is any one of strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), and strontium ranelate (C₁₂H₆N₂O₈SSr₂); wherein the relative density of the strontium compound is about 65% to 100%.

Statement 44: The bioresorbable bone graft according to Statement 43, wherein the relative density of the strontium compound is about 80% to 95%.

Statement 45: The bioresorbable bone graft according to any one of Statement 43 or 44, wherein the bioresorbable bone graft is in the form of pellets.

Statement 46: A bioresorbable bone graft, the bioresorbable bone graft is a calcium compound, the calcium compound is any one of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₇O), hydroxyapatite (Ca₅(PO₄)₃(OH), calcium carbonate (CaCO₃), calcium oxide (CaO) and calcium silicate (CaSiO₃); wherein the relative density of the calcium compound is about 65% to 100%.

Statement 47: The bioresorbable bone graft according to Statement 46, wherein the relative density of the calcium compound is about 80% to 95%.

Statement 48: The bioresorbable bone graft according to any one of Statement 46 or 47, wherein the bioresorbable bone graft is in the form of pellets.

Statement 49: A bioresorbable bone graft product for use in bioresorbable bone graft implantation, comprising: a liquid, the liquid is derived from non-biological origins; a binder, the binder is a synthetic organic polymer or a natural organic polymer; a powder mixture, and the powder mixture comprises a powder formed from the bioresorbable bone graft according to any one of Statement 43 to 48.

Statement 50: The bioresorbable bone graft product according to Statement 49, wherein the liquid derived from non-biological origin comprises one or more of Hank's balanced salt solution, phosphate buffered saline, sodium hydrogen phosphate (Na₂HPO₄) solution, dicalcium phosphate anhydrous (CaHPO₄) solution, strontium ranelate (C₁₂H₆N₂O₈SSr₂) solution, water, and stimulated body fluid.

Statement 51: The bioresorbable bone graft product according to any one of Statement 49 or 50, wherein the synthetic organic polymer comprises one or more of polyactic acid (PLA), poly(lactic-co-glycolic acid) (PLGA), poly-L-lactide (PLLA), poly-DL-lactic acid (PDLLA), polycaprolactone (PCL), polyethylene glycol, poly(α-hydroxy ester), poly(N-isopropyl acrylamide), pluronic block copolymers, and carboxymethyl cellulose.

Statement 52: The bioresorbable bone graft product according to any one of Statement 49 to 51, wherein the natural organic polymer comprises one or more of agarose gel, alginate, carrageenan, chitosan, collagen, fibrinogen, gelatin, hyaluronic acid, and starch.

Statement 53: The bioresorbable bone graft product according to any one of Statement 49 to 52, wherein the powder mixture further comprising a hardening agent, the hardening agent comprises one or more of calcium sulfate anhydrite (CaSO₄), calcium sulfate hemihydrate (CaSO₄ 0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH), calcium carbonate (CaCO₃), magnesium carbonate (MgCO₃), strontium carbonate (SrCO₃), sodium hydrogen phosphate (Na₂HPO₄) and the bioglass.

Statement 54: A method for preparing and implanting the bioresorbable bone graft paste, comprising a) mix the powder mixture, the binder and the liquid according to any one of Statement 49 to 53 to form a bioresorbable bone graft paste; b) deliver the bioresorbable bone graft paste to one or more implantation locations.

The foregoing descriptions of specific compositions and methods of the present disclosure have been presented for purposes of illustration and description. They are not intended to be exhaustive or to limit the disclosure to the precise compositions and methods disclosed and obviously many modifications and variations are possible in light of the above teaching. The examples were chosen and described in order to best explain the principles of the disclosure and its practical application, to thereby enable others skilled in the art to best utilize the disclosure with various modifications as are suited to the particular use contemplated. It is intended that the scope of the disclosure be defined by the claims appended hereto and their equivalents.

What is claimed is:

1. A solid solution for use in bone regeneration, comprising:
at least one divalent cation, wherein the divalent cation is calcium ion and/or strontium ion,
at least one anion, and the at least one anion comprises one or more of sulfate, phosphate, carbonate, and silicate;
wherein the relative density of the solid solution is about 65% to 100%.

2. The solid solution of claim 1, wherein the solid solution further comprising at least one additional divalent cation, the additional divalent cation comprises one or more of magnesium ion, barium ion, and zinc ion.

3. The solid solution of claim 1, wherein the relative density of the solid solution is about 80% to 95%.

4. The solid solution of claim 1, wherein the molar ratio of the cation to the anion is 1 to 1.5.

5. The solid solution of claim 1, wherein the divalent cations are calcium ion and strontium ion, and the molar ratio of calcium ion and strontium ion is 1 to 33.

6. The solid solution of claim 5, wherein the molar ratio of calcium ion and strontium ion is 6 to 20.

7. The solid solution of claim 1, wherein the solid solution is in the form of pellets.

8. A bioresorbable bone graft product for use in bioresorbable bone graft implantation, comprising:
a liquid, the liquid is derived from non-biological origin;
a binder, the binder is a synthetic organic polymer or a natural organic polymer;
a powder mixture, and the powder mixture comprises a solid solution powder, the solid solution powder comprises at least one divalent cation and at least one anion, wherein the at least one divalent cation is calcium ion and/or strontium ion, and the at least one anion comprises one or more of sulfate, phosphate, carbonate, and silicate; wherein the relative density of the solid solution powder is about 65% to 100%.

9. The bioresorbable bone graft product of claim 8, wherein the solid solution powder further comprising at least one additional divalent cation, the additional divalent cation comprises one or more of magnesium ion, barium ion, and zinc ion.

10. The bioresorbable bone graft product of claim 8, wherein the relative density of the solid solution powder is about 80% to 95%.

11. The bioresorbable bone graft product of claim 8, wherein the molar ratio of the cation to the anion is 1 to 1.5 in the solid solution powder.

12. The bioresorbable bone graft product of claim 8, wherein the divalent cations are calcium ion and strontium ion, and the molar ratio of calcium ion and strontium ion is 1 to 33 in the solid solution powder.

13. The bioresorbable bone graft product of claim 12, wherein the molar ratio of calcium ion and strontium ion is 6 to 20 in the solid solution powder.

14. The bioresorbable bone graft product of claim 8, wherein the liquid derived from non-biological origin comprises one or more of Hank's balanced salt solution, phosphate buffered saline, sodium hydrogen phosphate solution, dicalcium phosphate anhydrous solution, strontium ranelate solution, water, and stimulated body fluid.

15. The bioresorbable bone graft product of claim 8, wherein the synthetic organic polymer comprises one or more of polyactic acid (PLA), poly(lactic-co-glycolic acid) (PLGA), poly-L-lactide (PLLA), poly-DL-lactic acid (PDLLA), polycaprolactone (PCL), polyethylene glycol, poly(α-hydroxy ester), poly(N-isopropyl acrylamide), pluronic block copolymers, and carboxymethyl cellulose.

16. The bioresorbable bone graft product of claim 8, wherein the natural organic polymer comprises one or more of agarose gel, alginate, carrageenan, chitosan, collagen, fibrinogen, gelatin, hyaluronic acid, and starch.

17. The bioresorbable bone graft product of claim 8, wherein the powder mixture further comprising a hardening agent, the hardening agent comprises one or more of calcium sulfate hemihydrate, calcium sulfate dihydrate, calcium sulfate anhydrite, monocalcium phosphate, dicalcium phosphate, tricalcium phosphate, tetracalcium phosphate, octacalcium phosphate, hydroxyapatite, calcium carbonate, magnesium carbonate, strontium carbonate, sodium hydrogen phosphate, and the bioglass.

18. A method for preparing and implanting the bioresorbable bone graft paste, comprising:
a) mix the powder mixture, the binder and the liquid of claim 8 to form a bioresorbable graft paste;
b) deliver the bioresorbable bone graft paste to one or more implantation locations.
